(19) United States
(12) Patent Application Publication
(10) Pub. No.: US 2024/0065555 A1
(43) Pub. Date: Feb. 29, 2024

(54) SPATIOTEMPORAL ANTIALIASING IN PHOTOACOUSTIC COMPUTED TOMOGRAPHY

(71) Applicant: California Institute of Technology, Pasadena, CA (US)

(72) Inventors: Peng Hu, Pasadena, CA (US); Lihong Wang, Arcadia, CA (US)

(21) Appl. No.: 18/450,597

(22) Filed: Aug. 16, 2023

Related U.S. Application Data
(63) Continuation-in-part of application No. 17/090,752, filed on Nov. 5, 2020.
(60) Provisional application No. 63/398,332, filed on Aug. 16, 2022.
(60) Provisional application No. 62/931,024, filed on Nov. 5, 2019.

(51) Int. Cl. A61B 5/00; G06T 11/00

(52) U.S. Cl. CPC A61B 5/0095; A61B 5/725; G06T 11/008; G06T 2210/41

(57) ABSTRACT

Among the various aspects of the present disclosure is the provision of systems and methods of imaging using photoacoustic computed tomography.

### CROSS-REFERENCES TO RELATED APPLICATIONS

This application claims priority to and benefit of U.S. Provisional Patent Application No. 63/398,332, and filed on Aug. 16, 2022, which is hereby incorporated by reference in its entirety and for all purposes. This application is a continuation-in-part of U.S. application Ser. No. 17/090,752, filed Nov. 15, 2020, which claims priority to and benefit of U.S. Provisional Patent Application 62/931,024, filed Nov. 5, 2019, each of which is incorporated by reference in its entirety and for all purposes.

### FEDERALLY SPONSORED RESEARCH OR DEVELOPMENT

This invention was made with government support under Grant No(s). NS102213 & NS099717 & EB029823 & CA220436 awarded by the National Institutes of Health. The government has certain rights in the invention.

### FIELD

Certain aspects generally pertain to photoacoustic imaging and, more specifically, to photoacoustic computed tomography systems and methods.

### BACKGROUND

Photoacoustic tomography provides high-resolution images beyond the optical diffusion limit by combining optical absorption contrast and ultrasonic spatial resolution. By converting highly scattered photons into ultrasonic waves, which are much less scattered than light in biological tissues, photoacoustic tomography can be a useful technique for forming high-resolution images of the optical properties of biological tissues.

### SUMMARY

Certain aspects pertain to photoacoustic computed tomography with spatiotemporal antialiasing techniques as can be used, for example, to image biological tissues.

In some embodiments, a photoacoustic computed tomography method involves acquiring photoacoustic data recorded by one or more data acquisition devices of a photoacoustic computed tomography system. The method may further involve applying location-based temporal filtering to the photoacoustic data acquired, wherein applying the location-based temporal filtering is on a sub-domain by sub-domain basis for a plurality of sub-domains, the plurality of sub-domains when aggregated forming an entire imaging domain. The method may further involve reconstructing one or more photoacoustic images from the filtered photoacoustic data.

In some examples, the method further involves applying spatial interpolation after applying the location-based temporal filtering. In some examples, the spatial interpolation is performed on the sub-domain by sub-domain basis for the plurality of sub-domains. In some examples, the temporal filtering mitigates aliasing prior to the spatial interpolation. In some examples, applying the location-based temporal filtering comprises: determining an upper cutoff frequency for each sub-domain; and applying one or more lowpass filters associated with the upper cutoff frequency. In some examples, the one or more lowpass filters associated with the upper cutoff frequency comprise a plurality of lowpass filters each having a different cutoff frequency less than the upper cutoff frequency, and applying the location-based temporal filtering may involve: applying the plurality of lowpass filters to the photoacoustic data; and recentering the filtered photoacoustic data for different image subdomains. In some examples, wherein the upper cutoff frequency is selected such that a Nyquist criterion is satisfied for each sub-domain. In some examples, the upper cutoff frequency for a given sub-domain is determined based on relative locations of transducer elements, a center of the sub-domain, and points on a boundary of the sub-domain. In some examples, a point source is outside of a sub-domain, and wherein the upper cutoff frequency for the sub-domain is further based on relative locations of transducer elements, a center of the sub-domain and the point source.

In some examples, a general source causes reflections indicated in the photoacoustic data, and the method further involves: performing an initial reconstruction without anti-aliasing to generate a reconstructed image; obtaining a set of point source candidates based on the reconstructed image; dividing the entire imaging domain into a set of squares; randomly selecting one point source in each square of the set of squares as a point source; applying the location-based filtering for each sub-domain based on the selected point sources to generate an image; repeating the random selection of one point source in each square and applying the location-based filtering for each sub-domain at least one other time to obtain multiple images; and generating a final reconstructed image based on an average of the multiple images. In some examples, the initial reconstruction is performed using universal back projection. In some examples, the set of point source candidates is obtained by applying a threshold to the reconstructed image.

In some examples, universal back projection is used to reconstruct the one or more photoacoustic images.

In some embodiments, a non-transitory computer readable media is provided, where the non-transitory computer readable media, when read by one or more processors, is configured to perform operations that may involve acquiring photoacoustic data recorded by one or more data acquisition devices of a photoacoustic computed tomography system. The operations may further involve applying location-based temporal filtering to the photoacoustic data acquired, wherein applying the location-based temporal filtering is on a sub-domain by sub-domain basis for a plurality of sub-domains, the plurality of sub-domains when aggregated forming an entire imaging domain. The operations may further involve reconstructing one or more photoacoustic images from the filtered photoacoustic data.

In some examples, the operations further involve applying spatial interpolation after applying the location-based temporal filtering. In some examples, the spatial interpolation is performed on the sub-domain by sub-domain basis for the plurality of sub-domains. In some examples, the temporal filtering mitigates aliasing prior to the spatial interpolation.

In some examples, applying the location-based temporal filtering involves: determining an upper cutoff frequency for each sub-domain; and applying one or more lowpass filters associated with the upper cutoff frequency. In some examples, the one or more lowpass filters associated with the upper cutoff frequency comprise a plurality of lowpass filters each having a different cutoff frequency less than the upper cutoff frequency, and applying the location-based temporal filtering involves: applying the plurality of lowpass filters to the photoacoustic data; and recentering the filtered photoacoustic data. In some examples, the upper cutoff frequency is selected such that a Nyquist criterion is satisfied for each sub-domain. In some examples, the upper cutoff frequency for a given sub-domain is determined based on relative locations of transducer elements, a center of the sub-domain, and points on a boundary of the sub-domain. In some examples, a point source is outside of a sub-domain, and wherein the upper cutoff frequency for the sub-domain is further based on relative locations of transducer elements, a center of the sub-domain and the point source.

In some examples, a general source causes reflections indicated in the photoacoustic data, and the operations further involve: performing an initial reconstruction without anti-aliasing to generate a reconstructed image; obtaining a set of point source candidates based on the reconstructed image; dividing the entire imaging domain into a set of squares; randomly selecting one point source in each square of the set of squares as a point source; applying the location-based filtering for each sub-domain based on the selected point sources to generate an image; repeating the random selection of one point source in each square and applying the location-based filtering for each sub-domain at least one other time to obtain multiple images; and generating a final reconstructed image based on an average of the multiple images.

These and other features are described in more detail below with reference to the associated drawings.

### BRIEF DESCRIPTION OF THE DRAWINGS

FIG. 1 is a schematic diagram of components of a PACT system with spatiotemporal antialiasing, according to certain implementations.

FIG. 2 is signal flow diagram of signals communicated between components of a PACT system with spatiotemporal antialiasing, according to implementations.

FIG. 3 is a flowchart depicting operations of a PACT method with spatiotemporal antialiasing that implements one or more antialiasing techniques, according to an implementation.

FIGS. 4A-4C depict configurations of point sources within and outside of a given sub-domain according to some implementations.

FIG. 5 is a flowchart of an example process for reconstructing a photoacoustic image according to some implementations.

FIG. 6 is a flowchart of another example process for reconstructing a photoacoustic image according to some implementations.

FIG. 7 is a flowchart of an example process for reconstructing a photoacoustic image given a general source according to some implementations.

FIGS. 8, 9, 10, and 11 depict results of an example technique for reconstructing a photoacoustic image according to some implementations.

These and other features are described in more detail below with reference to the associated drawings.

### DETAILED DESCRIPTION

Different aspects are described below with reference to the accompanying drawings. The features illustrated in the drawings may not be to scale. In the following description, numerous specific details are set forth in order to provide a thorough understanding of the presented embodiments. The disclosed embodiments may be practiced without one or more of these specific details. In other instances, well-known operations have not been described in detail to avoid unnecessarily obscuring the disclosed embodiments. While the disclosed embodiments will be described in conjunction with the specific embodiments, it will be understood that it is not intended to limit the disclosed embodiments.

I. Introduction to Photoacoustic Computed Tomography (PACT)

Photoacoustic computed tomography (PACT) is an imaging modality that ultrasonically images optical contrast via the photoacoustic effect. PACT can provide tomographic images (e.g., two-dimensional (2D) section(s) and/or 3D volume(s) constructed from 2D sections) of biological tissues and other samples. By converting highly scattered photons into ultrasonic waves, which are much less scattered than light in biological tissues, PACT can form high-resolution images of the optical properties of these tissues at different depths. Some examples of photoacoustic imaging of biological tissues can be found in Kruger, R. A., Kuzmiak, C. M., Lam, R. B., Reinecke, D. R., Del Rio, S. P. and Steed, D., “Dedicated 3D photoacoustic breast imaging,” Med. Phys., vol. 40, no. 11 (2013) (hereinafter “Kruger”), Tzoumas, S., et al., “Eigenspectra optoacoustic tomography achieves quantitative blood oxygenation imaging deep in tissues,” Nat. Commun., vol. 7 (2016), Dean-Ben, X. L. et al., “Functional optoacoustic neuro-tomography for scalable whole-brain monitoring of calcium indicators,” Light Sci. Appl., vol. 5, no. 12, p. e16201, (2016), Li, L. et al., “Single-impulse panoramic photoacoustic computed tomography of small-animal whole-body dynamics at high spatiotemporal resolution,” Nat. Biomed. Eng., vol. 1, no. 5, p. 0071 (2017), Matsumoto, Y., et al., “Label-free photoacoustic imaging of human palmar vessels: a structural morphological analysis,” Sci. Rep., vol. 8, no. 1, p. 786 (2018), Lin, L. et al., “Single-breath-hold photoacoustic computed tomography of the breast,” Nat. Commun., vol. 9, no. 1, p. 2352 (2018), Li, L., “Small near-infrared photochromic protein for photoacoustic multi-contrast imaging and detection of protein interactions in vivo,” Nat. Commun., vol. 9, no. 1, p. 2734 (2018), and Wu, Z. et al., “A microrobotic system guided by photoacoustic computed tomography for targeted navigation in intestines in vivo,” Sci. Robot., vol. 4, no. 32, p. 0613, (2019), which are hereby incorporated by reference in their entireties. Some examples of PACT methods and systems can be found in U.S. patent application Ser. No. 16/798,204, titled “PHOTOACOUSTIC COMPUTED TOMOGRAPHY (PACT) SYSTEMS AND METHODS,” filed on Feb. 21, 2020; PCT publication WO2018102446, titled “SINGLE-IMPULSE PANORAMIC PHOTOACOUSTIC COMPUTED TOMOGRAPHY (SIP-PACT), filed on Nov. 29, 2017; U.S. Patent Publication US 2016/0262628, titled “PHOTOACOUSTIC COMPUTED TOMOGRAPHY WITH AN ACOUSTIC REFLECTOR,” filed on Nov. 12, 2014; which are hereby incorporated by reference in their entireties.

PACT methods generally include a data acquisition phase and an image reconstruction phase. During the data acquisition phase, photon-induced acoustic waves (sometimes referred to herein as “photoacoustic waves”) are detected by an ultrasonic transducer array or its scanning equivalent, such as a full-ring ultrasonic transducer array, a linear array, or two-dimensional array. During the image reconstruction phase, the detected acoustic signals are used to reconstruct the sample's optical absorption via inverse reconstruction algorithms. Some examples of inverse reconstruction algorithms that can be used include: (i) forward-model-based iterative methods, (ii) time-reversal methods, and (iii) the universal back-projection (UBP) method. Examples of the universal back-projection (UBP) method can be found in Kruger, Li, L. et al., “Single-impulse panoramic photoacoustic computed tomography of small-animal whole-body dynamics at high spatiotemporal resolution,” Nat. Biomed. Eng., vol. 1, no. 5, p. 0071 (2017), Lin, L. et al., “Single-breath-hold photoacoustic computed tomography of the breast,” Nat. Commun., vol. 9, no. 1, p. 2352 (2018), Xu M. and Wang, L. V., “Universal back-projection algorithm for photoacoustic computed tomography,” Phys. Rev. E, vol. 71, no. 1, p. 016706 (2005), Song, L., Maslov, K. I., Bitton, R., Shung, K. K., and Wang, L. V., “Fast 3-D dark-field reflection-mode photoacoustic microscopy in vivo with a 30-MHz ultrasound linear array,” J. Biomed. Opt., vol. 13, no. 5, p. 054028 (2008), Dean-Ben, X. L. and Razansky, D., “Portable spherical array probe for volumetric real-time optoacoustic imaging at centimeter-scale depths,” Opt. Express, vol. 21, no. 23, pp. 28062-28071 (2013), and Pramanik, M., “Improving tangential resolution with a modified delay-and-sum reconstruction algorithm in photoacoustic and thermoacoustic tomography,” JOSA A, vol. 31, no. 3, pp. 621-627 (2014), which are hereby incorporated by reference in their entireties. Examples of forward-model-based iterative methods can be found in Wang, K., et al. “Investigation of iterative image reconstruction in three-dimensional optoacoustic tomography,” Phys. Med. Biol., vol. 57, no. 17, p. 5399 (2012), Huang, Chao et al., “Full-wave iterative image reconstruction in photoacoustic tomography with acoustically inhomogeneous media,” IEEE Trans. Med. Imaging, vol. 32, no. 6, pp. 1097-1110 (June 2013), Mitsuhashi, K., et al., “A forward-adjoint operator pair based on the elastic wave equation for use in transcranial photoacoustic computed tomography,” SIAM J. Imaging Sci., vol. 10, no. 4, pp. 2022-2048 (2017), Treeby, B. E. and Cox, B. T., “k-Wave: MATLAB toolbox for the simulation and reconstruction of photoacoustic wave fields,” J. Biomed. Opt., vol. 15, no. 2, p. 021314, (2010), Mitsuhashi, K., Wang, K. and Anastasio, M. A., “Investigation of the far-field approximation for modeling a transducer's spatial impulse response in photoacoustic computed tomography,” Photoacoustics, vol. 2, no. 1, pp. 21-32 (2014), Han, Y., Ntziachristos, V. and Rosenthal, A., “Optoacoustic image reconstruction and system analysis for finite-aperture detectors under the wavelet-packet framework,” J. Biomed. Opt., vol. 21, no. 1, p. 016002 (2016), Arridge, S., et al., “Accelerated high-resolution photoacoustic tomography via compressed sensing,” ArXiv Prepr., ArXiv160500133 (2016), Han, Y. et al., “Three-dimensional optoacoustic reconstruction using fast sparse representation,” Opt. Lett., vol. 42, no. 5, pp. 979-982 (2017), Schoeder, S. et al. “Optoacoustic image reconstruction: the full inverse problem with variable bases,” Proc. R. Soc. A., vol. 474, no. 2219, p. 20180369 (2018), and Matthews, T. P., Poudel, J., Li, L., Wang, L. V. and Anastasio, M. A., “Parameterized Joint Reconstruction of the Initial Pressure and Sound Speed Distributions for Photoacoustic Computed Tomography,” SIAM J. Imaging Sci., vol. 11, no. 2, pp. 1560-1588 (2018), which are hereby incorporated by reference in their entireties. Examples of time reversal methods can be found in Xu, Y. and Wang, L. V., “Time reversal and its application to tomography with diffracting sources,” Phys. Rev. Lett., vol. 92, no. 3, p. 033902 (2004), Treeby, B. E., Zhang, E. Z. and Cox, B., “Photoacoustic tomography in absorbing acoustic media using time reversal,” Inverse Probl., vol. 26, no. 11, p. 115003 (2010), Cox, B. T. and Treeby, B. E., “Artifact trapping during time reversal photoacoustic imaging for acoustically heterogeneous media,” IEEE Trans. Med. Imaging, vol. 29, no. 2, pp. 387-396 (2010), Treeby, B. E., Jaros, J. and Cox, B. T., “Advanced photoacoustic image reconstruction using the k-Wave toolbox,” in Photons Plus Ultrasound: Imaging and Sensing 2016, vol. 9708, p. 97082P (2016), and Ogunlade, O. et al., “In vivo three-dimensional photoacoustic imaging of the renal vasculature in preclinical rodent models,” Am. J. Physiol.-Ren. Physiol., vol. 314, no. 6, pp. F1145-F1153 (2017), which are hereby incorporated by reference in their entireties. Examples of universal back-projection (UBP) method can be found in Kruger, Li, L. et al., “Single-impulse panoramic photoacoustic computed tomography of small-animal whole-body dynamics at high spatiotemporal resolution,” Nat. Biomed. Eng., vol. 1, no. 5, p. 0071 (2017), Lin, L. et al., “Single-breath-hold photoacoustic computed tomography of the breast,” Nat. Commun., vol. 9, no. 1, p. 2352 (2018), Xu M. and Wang, L. V., “Universal back-projection algorithm for photoacoustic computed tomography,” Phys. Rev. E, vol. 71, no. 1, p. 016706 (2005), Song, L., Maslov, K. I., Bitton, R., Shung, K. K., and Wang, L. V., “Fast 3-D dark-field reflection-mode photoacoustic microscopy in vivo with a 30-MHz ultrasound linear array,” J. Biomed. Opt., vol. 13, no. 5, p. 054028 (2008), Dean-Ben, X. L. and Razansky, D., “Portable spherical array probe for volumetric real-time optoacoustic imaging at centimeter-scale depths,” Opt. Express, vol. 21, no. 23, pp. 28062-28071 (2013), and Pramanik, M., “Improving tangential resolution with a modified delay-and-sum reconstruction algorithm in photoacoustic and thermoacoustic tomography,” JOSA A, vol. 31, no. 3, pp. 621-627 (2014), which are hereby incorporated by reference in their entireties.

PACT based on a full-ring ultrasonic transducer array is widely used for small animal whole body and human organ imaging, thanks to its high in-plane resolution and full-view fidelity. Some discussion of PACT used to for small animal whole body and/or human organ imaging can be found in Li, L. et al., “Single-impulse panoramic photoacoustic computed tomography of small-animal whole-body dynamics at high spatiotemporal resolution,” Nat. Biomed. Eng., vol. 1, no. 5, p. 0071 (2017), Xu, Y., Xu, M. and Wang, L. V., “Exact frequency-domain reconstruction for thermoacoustic tomography. II. Cylindrical geometry,” IEEE Trans. Med. Imaging, vol. 21, no. 7, pp. 829-833,2002, which are hereby incorporated by reference in their entireties. To help avoid any artifacts occurring in image reconstruction in PACT, the ultrasonic transducer array should provide dense spatial sampling around the object to satisfy the Nyquist sampling theorem. That is, the spatial sampling interval on the specimen surface should be less than half of the lowest detectable acoustic wavelength. Otherwise, artifacts may appear in image reconstruction, a problem called spatial aliasing. In practice, due to the high cost of a transducer array with a large number of elements or limited scanning time, spatially sparse sampling is common.

PACT systems and methods with spatiotemporal antialiasing described herein may mitigate artifacts caused by spatial under-sampling without having to physically increase the number of elements in the transducer array to satisfy Nyquist sampling criterion. Moreover, in some implementations, the PACT systems and/or methods with spatiotemporal antialiasing may compensate for under-sampling without substantially compromising image resolution. PACT systems/methods with spatiotemporal antialiasing may be advantageous in reducing equipment costs by being able to utilize less expensive ultrasonic transducer arrays that sparsely sample while still avoiding artifacts and maintaining high resolution in photoacoustic images.

In the following sections, the sources of spatial aliasing in a full-ring geometry PACT and linear array PACT and applications to other array geometries are discussed. The PACT systems and methods with spatiotemporal antialiasing described here implement one or more techniques that may mitigate artifacts caused by spatial aliasing without physically increasing the number of elements in an ultrasonic transducer array. Moreover, using the techniques described herein, spatial resolution may be maintained even while artifacts are mitigated.

Based on spatiotemporal analysis, two sources of spatial aliasing are: spatial sampling and image reconstruction. To classify different cases of spatial aliasing based on locations (of source points and reconstruction locations), three zones are defined in the imaging domain of the ultrasonic transducer array: (1) a detection zone S₀, (2) a one-way Nyquist zone S₁, and (3) a two-way Nyquist zone S₂. Zone S₁ is contained in S₀, while S₂ is contained in S₁. Spatial aliasing in spatial sampling does not appear for objects inside S₁, but appears for objects outside S₁. Spatial aliasing in image reconstruction does not appear for objects and reconstruction locations inside S₂, but appears for other combinations of objects and reconstruction locations. Zone S₂ is the original aliasing-free region, which can be extended to S₁ by one or more antialiasing techniques described herein. The aliasing free region can also be further extended from S₁ to S₁′ by one or more antialiasing techniques described herein.

Two categories of antialiasing techniques include: spatial interpolation and temporal filtering. Spatial interpolation can be used to eliminate spatial aliasing in image reconstruction without compromising the spatial resolution, effectively extending the alias-free zone from S₂ to S₁. Temporal filtering that uses an upper cutoff frequency based on Nyquist sampling criterion can remove aliasing in spatial sampling, while compromising the spatial resolution in the affected regions. Thus, this combination of spatial interpolation with temporal filtering using the upper cutoff frequency based on Nyquist sampling criterion removes spatial aliasing in both spatial sampling and image reconstruction, while compromising the spatial resolution.

In certain aspects, as a balance between spatial antialiasing and high resolution, location-based temporal filtering is performed, where location-based temporal filtering is applied on a sub-domain by sub-domain basis for a set of sub-domains that, in aggregate, form the entire imaging domain. Because temporal filtering is performed on a sub-domain by sub-domain basis, the location dependency of the Nyquist criterion at different regions of the imaging domain can be accounted for. In other words, because the Nyquist criterion may vary with different locations of transducer elements and/or different locations of a source, location-based temporal filtering may allow spatial resolution to be maintained by satisfying the Nyquist criterion for the filtered photoacoustic data. After performing location-based temporal filtering, in some embodiments, spatial interpolation may be performed. The spatial interpolation may interpolate between physical transducer elements. Note that spatial interpolation may be performed in a location-based manner, e.g., on a sub-domain by sub-domain basis. By performing location-based temporal filtering and location-based spatial interpolation on a sub-domain by sub-domain basis, the techniques disclosed herein may substantially or even completely remove aliasing-related artifacts while maintaining spatial resolution. For example, using the techniques disclosed herein, “true” signals associated with real image features (e.g., in vivo tissue features, blood vessels, etc.) may be presented with relatively high spatial resolution while artifacts are substantially or completely removed.

II. PACT Systems and Methods with Spatiotemporal Antialiasing

Spatial aliasing may be generally classified into two categories: (1) aliasing in spatial sampling and (2) aliasing in image reconstruction. In certain implementations, PACT systems and methods with spatiotemporal antialiasing described herein implement antialiasing techniques including: (i) spatial interpolation and/or (ii) location-dependent temporal filtering that is performed on a sub-domain by sub-domain basis. Spatial interpolation can mitigate (reduce or eliminate) spatial aliasing in image reconstruction.

Although the spatiotemporal antialiasing techniques are described in this section with reference to a transducer array having a circular geometry and a transducer array having a linear geometry, it would be understood that the disclosure is not so limiting, and that these techniques would apply to other array geometries. For example, based on the basic 1D circular and linear geometries, one can apply 2D/3D geometries, such as, a planar geometry and the spherical geometry, through decomposition. Moreover, although the antialiasing techniques are sometimes described being implementing with the universal back-projection (UBP) described in Xu, M. and Wang, L. V., “Universal back-projection algorithm for photoacoustic computed tomography,” Phys. Rev. E, vol. 71, no. 1, p. 016706 (2005), it would be understood that other inverse reconstruction techniques can be used, such as those referenced in Section I. In addition, the antialiasing techniques described herein may apply to other image reconstruction methods. For example, spatial interpolation may be used in time reversal methods to generate a dense enough grid for numerical computation [20]—[23]. Furthermore, location-dependent temporal filtering may be incorporated into a wave propagation model and be used in time reversal methods and/or iterative methods to mitigate aliasing in spatial sampling according to other aspect.

### Photoacoustic Image Reconstruction

In a homogeneous medium, a photoacoustic wave can be expressed as:

$$p ( r , t ) = \frac{1}{4 π {c}^{2}} \frac{\partial }{\partial t} ( \frac{1}{ct} {\int }_{V} {dr}^{′} {p}_{0} ( {r}^{′} ) δ ( t - \frac{‖ {r}^{′} - r ‖}{c} ) ) . \qquad ( Eqn . 1 )$$

A detailed discussion of the expression in Eqn. 1 can be found Wang, L. V. and Wu, H., Biomedical optics: principles and imaging. John Wiley & Sons (2012), Zhou, Y., Yao, J. and Wang, L. V., “Tutorial on photoacoustic tomography,” J. Biomed. Opt., vol. 21, no. 6, p. 061007, 2016, which are hereby incorporated by reference in their entireties. In Eqn. 1, p(r, t) is the pressure at location r and time t, c is the speed of sound, V is the volumetric space occupied by the tissue, and p₀(r′) is the initial pressure at r′. Eqn. 1 can be rewritten as:

$$p ( r , t ) = \frac{1}{4 π {c}^{2}} {\int }_{V} \frac{{p}_{0} ( {r}^{′} )}{‖ {r}^{′} - r ‖} \frac{\partial }{\partial t} δ ( t - \frac{‖ {r}^{′} - r ‖}{c} ) ) {dr}^{′} . \qquad ( Eqn . 2 )$$

Discretizing Eqn. 2 in space, provides:

$$p ( {r}_{n} , t ) = \frac{1}{4 π {c}^{2}} {\sum }_{m = 1}^{M} {v}_{m} \frac{{p}_{0} ( {r}_{m}^{′} )}{‖ {r}_{m}^{′} - {r}_{n} ‖} \frac{\partial }{\partial t} δ ( t - \frac{‖ {r}_{m}^{′} - {r}_{n} ‖}{c} ) , n = 1 , 2 , … , N . \qquad ( Eqn . 3 )$$

Eqn. 3 refers to M source points distributed at =1, 2, . . . , M, and N point detection elements distributed at rₙ, n=1, 2, . . . , N. The term vₘ in is the volume of the m-th source point. The response of an ultrasonic transducer array can be described as:

\hat{p}(rₙ,t)=p(rₙ,t)*ₜhₑ(t),n=1,2, . . . ,N. (Eqn. 4)

Here, \hat{p}(rₙ,t) is the pressure impinging on the n-th point detection element (transducer element) at time t, and hₑ(t) is the ultrasonic transducer's electric impulse response. Substituting Eqn. 3 into Eqn. 4, the following is obtained:

$$\hat{p} ( {r}_{n} , t ) = \frac{1}{4 π {c}^{2}} {\sum }_{m = 1}^{M} {v}_{m} \frac{{p}_{0} ( {r}_{m}^{′} )}{‖ {r}_{m}^{′} - {r}_{n} ‖} {h}_{e}^{′} ( t - \frac{‖ {r}_{m}^{′} - {r}_{n} ‖}{c} ) . \qquad ( Eqn . 5 )$$

The term

$${h}_{e}^{′} ( t - \frac{‖ {r}_{m}^{′} - {r}_{n} ‖}{c} )$$

is a function of both time and space, where the first prime denotes a temporal derivative.

When photoacoustic signals received by the ultrasonic transducer array are digitized by one or more data acquisition systems (DAQs), an antialiasing filter (e.g. one or more low-pass filters) with an upper cutoff frequency selected for a sufficiently high temporal sampling rate may be used to mitigate temporal aliasing in certain implementations. For simplicity, the time variable is assumed to be continuous and spatial variables are discretized, which allows for a discussion of spatial sampling (sometimes referred to herein as “SS”). For different transducer detection geometries (e.g., planar, spherical, and cylindrical surfaces of transducer array), image mapping the initial pressure p₀(r″) using the Universal back-projection (UBP) algorithm found in Xu, M. and Wang, L. V., “Universal back-projection algorithm for photoacoustic computed tomography,” Phys. Rev. E, vol. 71, no. 1, p. 016706 (2005), provides:

$${p}_{0} ( {r}^{″} ) = {\int }_{{Ω}_{0}} b ( r , t = \frac{‖ {r}^{″} - r ‖}{c} ) \frac{d Ω}{{Ω}_{0}} , \qquad ( Eqn . 6 )$$

where the back-projection term,

$$b ( r , t ) = 2 p ( r , t ) - 2 t \frac{\partial p ( r , t )}{\partial t} , d Ω = \frac{d S}{{‖ {r}^{″} - r ‖}^{2}} \frac{{n}_{S} ( r ) \cdot ( {r}^{″} - r )}{‖ {r}^{″} - r ‖} ,$$

is the solid angle for the detection element at r with respect to reconstruction location r″, dS is the detection element surface area, and n_{S}(r) is the ingoing normal vector. The total solid angle is denoted as Ω₀. The actual or true pressure p(r,t) may be approximated by the detected pressure \hat{p}(rₙ,t), leading to the following discretized form of Eqn. 6:

$${\hat{p}}_{0} ( {r}^{″} ) \approx {\sum }_{n = 1}^{N} {w}_{n} \hat{b} ( {r}_{n} , t = \frac{‖ {r}^{″} - {r}_{n} ‖}{c} ) . \qquad ( Eqn . 7 )$$

Here. \hat{p}₀(r″) is the reconstructed initial pressure and

$$\hat{b} ( {r}_{n} , t ) = 2 \hat{p} ( {r}_{n} , t ) - 2 t \frac{\partial \hat{p} ( {r}_{n} , t )}{\partial t}$$

is the back-projection term computed from the detected pressure by the ultrasonic transducer array. The weights wₙ=1, 2, . . . , N come from dΩ/Ω₀ in Eqn. 6.
Substituting Eqn. 5 into Eqn. 7, the reconstructed initial pressure can be determined by:

$${\hat{p}}_{0} ( {r}^{″} ) \approx {\sum }_{n = 1}^{N} {w}_{n} {\sum }_{m = 1}^{M} {v}_{m} \frac{{p}_{0} ( {r}_{m}^{′} )}{‖ {r}_{m}^{′} - {r}_{n} ‖} \times ( 1 - ( t + \frac{‖ {r}_{m}^{′} - {r}_{n} ‖}{c} \frac{\partial }{\partial t} ) {h}_{e}^{′} ( \frac{‖ {r}^{″} - {r}_{n} ‖}{c} - \frac{‖ {r}_{m}^{′} - {r}_{n} ‖}{c} ) , {r}^{″} ϵ D \qquad ( Eqn . 8 )$$

PACT Systems and Methods with Spatiotemporal Antialiasing

In certain aspects, PACT systems and methods with spatiotemporal antialiasing mitigate (substantially reduce or eliminate) spatial aliasing in image reconstruction using spatial interpolation and mitigate spatial aliasing in spatial sampling using a location-dependent temporal filtering technique. The location-based temporal filtering and/or spatial interpolation may be implemented on a sub-domain by sub-domain basis where a set of sub-domains, in aggregate, form an entire imaging domain. By performing temporal filtering and/or spatial interpolation on a sub-domain by sub-domain basis that accounts for differences in the Nyquist criterion at different regions of the imaging domain, aliasing artifacts may be completely or nearly completely removed without decreasing spatial resolution.

Spatial aliasing in PACT has two sources: spatial sampling and image reconstruction. The spatial aliasing in spatial sampling and image reconstruction may be explained by analyzing the step size of

$$\frac{‖ {r}_{m}^{′} - {r}_{n} ‖}{c}$$

### in Eqn. 5 and

$$\frac{‖ {r}^{″} - {r}_{n} ‖}{c} - \frac{‖ {r}_{m}^{′} - {r}_{n} ‖}{c}$$

in Eqn. 8, respectively. An imaging domain D can be divided into sub-domains. These two terms may then be analyzed and anti-aliasing may be performed based on these two terms for each sub-domain. Note that, in general, image reconstruction is discussed herein in two dimensions and for rectangular sub-domains. However, the techniques disclosed herein may be extended to three-dimensional image reconstruction and/or for sub-domains having other shapes, such as circular, elliptical, etc. For a subdomain D_{sub} having size Iₓ×I_{y} and centered at r_{c,sub}′, the time t may be shifted to t′ according to

$${t}^{′} = t - \frac{‖ {r}_{c , sub}^{′} - {r}_{n} ‖}{c} .$$

The reconstructed pressure may be determined for a given sub-domain by:

$${\hat{p}}_{Dsub} ( {r}_{n} , {t}^{′} ) = p ( {r}_{n} , {t}^{′} + \frac{‖ {r}_{c , sub}^{′} - {r}_{n} ‖}{c} ) , n = 1 , 2 , … N \qquad ( Eqn . 9 )$$

The equation given above corresponds to a temporal recentering of signals based on the transducer elements' distances to the subdomain center r_{c,sub}′. After recentering, the signals originating from r_{c,sub}′ arrive at all detectors at time 0, and the exact range of interest for t′ is dynamically determined for each sub-domain. Note that this recentering minimizes temporal filtering and thus image blurring, which in turn reduces aliasing artifacts. Substituting Eqn. 5 into Eqn. 9 leads to:

$${\hat{p}}_{Dsub} ( {r}_{n} , {t}^{′} ) = \frac{1}{4 π {c}^{2}} {\sum }_{m = 1}^{M} {v}_{m} \frac{{p}_{0} ( {r}_{m}^{′} )}{‖ {r}_{m}^{′} - {r}_{n} ‖} \times {h}_{e}^{′} ( {t}^{′} - \frac{‖ {r}_{m}^{′} - {r}_{n} ‖}{c} - \frac{‖ {r}_{c , sub}^{′} - {r}_{n} ‖}{c} ) , n = 1 , 2 , … N \qquad ( Eqn . 10 )$$

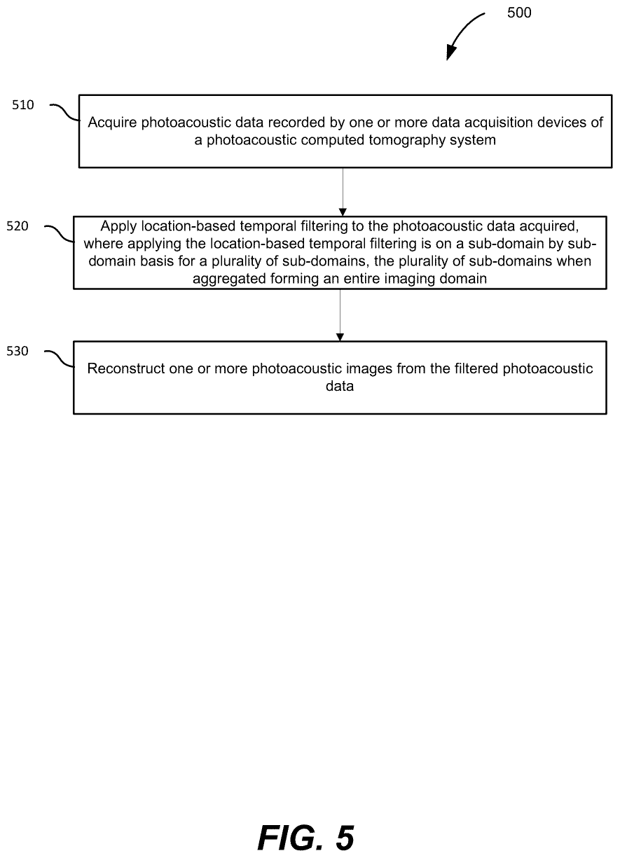

Eqn. 10, may be used to perform image reconstruction as described below in connection with FIGS. 5-7.

### Examples of PACT Systems and Methods with Spatiotemporal Antialiasing

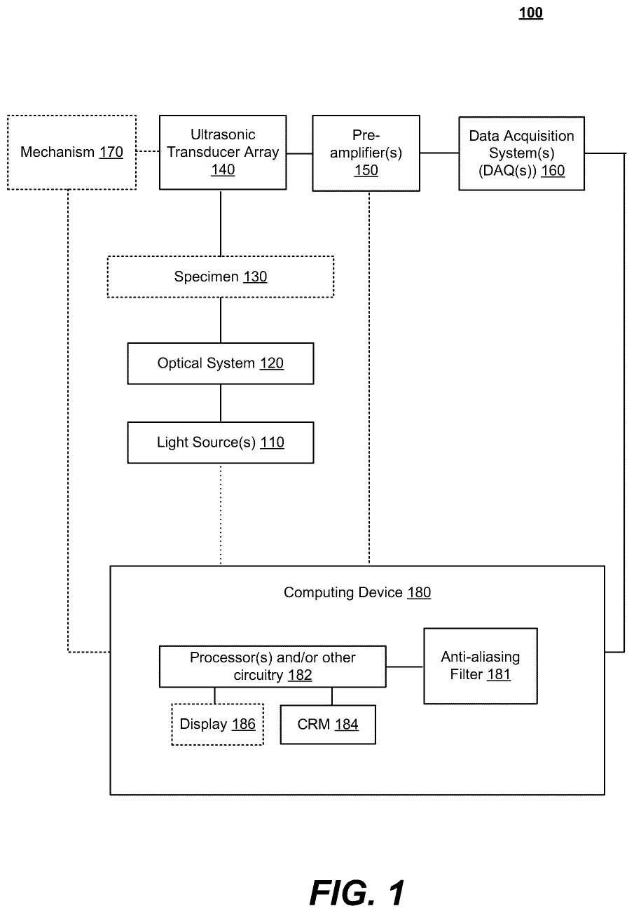

FIG. 1 is a schematic diagram of components of a PACT system with spatiotemporal antialiasing 100, according to certain implementations. The PACT system with spatiotemporal antialiasing 100 includes one or more light sources 110 (e.g., a pulsed laser) that provide illumination (e.g., generate pulsed or modulated light) and an optical system 120 having one or more optical components configured to propagate the illumination from the one or more light sources to a specimen 130 during operation. At the instant in time shown in FIG. 1, the specimen 130 is at a location at which it can receive the illumination during operation. It would be understood that at other times the specimen 130 may not be at this location as denoted by the dotted line.

The optical system 120 includes one or more optical components (e.g., lens(es), optical filter(s), mirror(s), beam steering device(s), beam-splitter(s), optical fiber(s), relay(s), and/or beam combiner(s)) configured to propagate and/or alter light from the one or more light sources 110 to provide the illumination to the specimen 130. The optical system 120 is in optical communication with the one or more light sources 110 to receive illumination during acquisition. In one aspect, the optical system 120 is configured to convert a light beam from the one or more light sources 110 into shaped illumination such as donut-shaped illumination (sometimes referred to herein as “donut illumination” or a “donut beam”). Donut-shaped illumination and other circular illumination may be generated by employing an engineered diffuser such as, e.g., an EDC15 diffuser made by RPC Photonics®.

In some implementations, a PACT system with spatiotemporal antialiasing includes one or more light sources (e.g., a pulsed laser) that can generate pulsed or modulated illumination such as, e.g., pulsed or modulated light at a near-infrared wavelength or a narrow band of near-infrared wavelengths. For example, a light source may be a pulsed laser that can generate near infrared pulses having a wavelength or narrow band of wavelengths in a range from about 700 nm to about 1000 nm. As another example, a light source may be a pulsed laser that can generate near infrared pulses having a wavelength or narrow band of wavelengths in a range from about 600 nm to about 1100 nm. In yet another example, a light source may be a pulsed laser that can generate near infrared pulses with a wavelength or narrow band of wavelengths greater than 760 nm. In yet another example, a light source may be a pulsed laser that can generate near infrared pulses with a wavelength or narrow band of wavelengths greater than 1000 nm. In another example, a light source may be a pulsed laser that can generate a 1064-nm laser beam. A commercially-available example of a suitable pulsed laser is the PRO-350-10, Quanta-Ray® laser with a 10-Hz pulse repetition rate and 8 ns-12 ns pulse width sold by Spectra-Physics®. The low optical attenuation of 1064 nm light or other near infrared light can be used to deeply penetrate to, e.g., a depth of 4 cm, into biological tissues. Imaging of biological tissues using near infrared light is discussed in Smith, A. M., Mancini, M. C. & Nie, S., “Bioimaging: second window for in vivo imaging,” Nat. Nanotechnol. 4, 710-711 (2009), which is hereby incorporated by reference in its entirety. Alternatively, a light source may be a continuous wave laser source that is chopped, modulated and/or gated. In implementations that include a light source in the form of a pulsed laser, the pulse repetition rate may be about 10-Hz in some cases, about 20-Hz in other cases, about 50-Hz in other cases, and about 100-Hz in other cases. In another case, the pulse repetition rate is in a range from about 10-Hz to about 100-Hz.

In one aspect, the one or more light sources of a PACT system with spatiotemporal antialiasing include a tunable narrow-band pulsed laser such as, e.g., one of a quantum cascade laser, an interband cascade laser, an optical parametric oscillator, or other pulsed laser that can be tuned to different narrow bands (e.g., a near-infrared band). In another aspect, the one or more light sources may include a pulsed laser of a single wavelength or approximately a single wavelength. In another aspect, the one or more light sources may include multiple lasers of the same type. For example, multiple lasers of the same type may be used where each of the lasers has a lower power. In another aspect, the one or more light sources may include a combination of different types of lasers. For example, an optical parametric oscillator combined with an Nd:YAG laser may be used in one implementation.

In one aspect, the optical system is configured to a light beam from the one or more light sources into shaped illumination, e.g., a donut beam that may be used to circumferentially illuminate the specimen. For example, the optical system may include an axicon lens (e.g., an axicon lens having 25 mm diameter and a 160° apex angle) followed by an engineered diffuser (e.g., EDC-10-A-2s made by RPC Photonics) to convert a light beam from the one or more light sources into a donut beam. The axicon lens may be positioned to receive a single laser beam propagated from a pulsed laser source and may be configured to convert the beam into a ring having a thickness and diameter and the engineered diffuser may be configured to expand the ring into a donut beam. The donut beam may be used to provide mass energy in homogenized, uniform illumination deep into tissue. An example of a technique for generating donut-shaped illumination can be found in U.S. patent application Ser. No. 16/464,958, titled “SINGLE-IMPULSE PANORAMIC PHOTOACOUSTIC COMPUTED TOMOGRAPHY (SIP-PACT),” and filed on Nov. 29, 2017, which is hereby incorporated by reference in its entirety.

Returning to FIG. 1, the PACT system with spatiotemporal antialiasing 100 also includes an ultrasonic transducer array 140 for sampling photoacoustic waves, an optional mechanism 170 for moving (translating and/or rotating) the ultrasonic transducer array 140, one or more pre-amplifiers 150 for boosting (increasing amplitude) photoacoustic signals communicated from the ultrasonic transducer array 140, one or more data acquisition systems (DAQs) 160 for digitizing the one or more boosted photoacoustic signal, and a computing device 180 for receiving the digitized photoacoustic data from the DAQ(s) 160. The computing device 180 includes an antialiasing filter 181 including one or more lowpass filters that can perform location-dependent temporal filtering of the digitized photoacoustic data communicated from the DAQ(s) 160. The computing device 180 also includes one or more processors and/or other circuitry 182 in electrical communication with the anti-aliasing filter 181 to receive photoacoustic data and configured to perform operations such as spatial interpolation and image reconstruction, an optional (denoted by dotted line) display 186 in electrical communication with the one or more processors and/or other circuitry 182, and a computer readable media 184. The computing device 180 may also communicate one or more control signals to the antialiasing filter 181 to control the upper cutoff frequency or to trigger temporally filtering. For example, the computing device 180 may execute instructions with logic for performing temporal filtering on a sub-domain by sub-domain basis, performing spatial interpolation on temporally-filtered photoacoustic data on a sub-domain by sub-domain basis, reconstructing spatiotemporal filtered images, or the like. The computing device may then send control signals to trigger the anti-aliasing filter to temporally filter the photoacoustic data. In one implementation, a GPU will be used for acceleration of both location-dependent temporal filtering and image reconstruction.

In certain implementations, the PACT system with spatiotemporal antialiasing includes an antialiasing filter (e.g., anti-aliasing filter 181 in FIG. 1) including one or more lowpass filters for location-dependent temporal filtering one or more photoacoustic signals from the DAQ(s). The antialiasing filter may include a single lowpass filter or a combination of the same type or different types of lowpass filters. Some examples of lowpass filters that be implemented include a Butterworth filter, a Chebyshev filter and a Bessel filter. The antialiasing filter is in electrical communication with the one or more DAQs of the PACT system to receive digitized photoacoustic data and remove frequency components with frequencies higher than an upper cutoff frequency f_{c} from digitized data communicated from the DAQ(s). The antialiasing filter may be a component of a computing device, a component of the controller, a component of one of the one or more DAQs, part of another system component, or a standalone device.

Returning to FIG. 1, the ultrasonic transducer array 140 is coupled or otherwise in acoustic communication with the specimen 130 to be able to receive one or more photoacoustic waves induced by the illumination from the one or more light sources 110 and output and/or record one or more photoacoustic signals. The one or more pre-amplifiers 150 are in electrical communication (directly or via other circuitry) with the ultrasonic transducer array 140 to receive one or more photoacoustic signals. The pre-amplifier(s) 150 can boost one or more photoacoustic signals received and output boosted photoacoustic signals. The DAQ(s) 160 is configured to process the photoacoustic signals, for example, digitize and/or record the digitized photoacoustic data. The DAQ(s) 160 may include at least one digitizer to digitize the signals.

During operation, the ultrasonic transducer array 140 is coupled to or otherwise in acoustic communication with the specimen 130 during signal acquisition. In some cases, an acoustic medium such as an acoustic gel, water, or other medium capable of conveying ultrasound pulses, is provided at least partially between the specimen 130 and the ultrasonic transducer array 140. In other cases, the acoustic medium may be omitted. The ultrasonic transducer array 140 is acoustically coupled to the specimen 130 to be able to detect photoacoustic waves induced by illumination and sample photoacoustic signals. These photoacoustic signals are indicative of the optical absorption of the specimen by the illumination. In one implementation, the PACT system with spatiotemporal antialiasing may also include a tank that is at least partially filed with acoustic medium such as a water tank (e.g., an acrylic water tank). The specimen being imaged may be located directly in the acoustic medium or in a portion of the tank that is submerged or otherwise located in the acoustic medium.

The ultrasonic transducer array 140 includes a plurality of N transducers (sometimes referred to herein as “transducer elements,” or “elements”) operable to collect photoacoustic signals, e.g., in parallel. Each transducer element has an aperture (e.g., a flat-rectangular aperture). The transducer elements have a height, a width, and a pitch. In one case, the pitch is about 1.35 mm. In one case, the width is 0.65 mm. In another case, the pitch is in a range of 1.20 mm to 1.50 mm. In another case, the height is about 5 mm. In another case, the height is in a range of 2 mm to 10 mm. The N transducer elements in the ultrasonic transducer array 140 are arranged in, e.g., a circular array such as a full-ring array, a linear array, or a two-dimensional array. In one implementation, a full-ring ultrasonic transducer array is employed, e.g., to be able to provide panoramic detection. In this case, the full-ring ultrasonic transducer array (e.g., a 512-element full-ring ultrasonic transducer) includes transducer elements distributed along the circumference of a ring having a diameter and an inter-element spacing. The ring diameter may be at least 220 mm in one aspect, may be at least 200 mm in one aspect, or may be at least 250 mm in one aspect. In one aspect, the ring diameter is in a range of about 150 mm to about 400 mm. The inter-element spacing may be less than or equal to about 1.0 mm in one aspect, less than or equal to 0.7 mm in one aspect, less than or equal to 1.5 mm in one aspect, or less than or equal to 2.0 mm in one aspect. In one aspect, the inter-element spacing is in a range of 0 mm to about 5 mm.

In one aspect, the ultrasonic transducer array includes one or more unfocused transducer elements. One or more of the unfocused transducer elements may have a diffraction angle of about 10 degrees in one case, a diffraction angle in a range of about 5 degrees to about degrees in another case, a diffraction angle of about 20 degrees in another case, a diffraction angle in a range of about 5 degrees to about 30 degrees in another case. In one case, each of the unfocused transducer elements has a central frequency in a range of 0.50 MHz to 2.25 MHz and a one-way bandwidth of more than 50%. In another aspect, each of the unfocused transducer elements has a central frequency in a range of 2.25 MHz to 10 MHz and a one-way bandwidth of more than 50%.

Returning to FIG. 1, the PACT system 100 includes an optional (denoted by dashed line) mechanism 170 coupled to or otherwise operably connected to the ultrasonic transducer array 140 to be able to move (translate and/or rotate) the ultrasonic transducer array 140 to one or more positions during operation, e.g., along an axis in one or both directions. For example, the mechanism 170 may be able to move ultrasonic transducer array 140 to two or more different positions along an axis (e.g., between z₁ and z₂ along a z-axis). In addition, the mechanism 170 may be able to hold the ultrasonic transducer array 140 at each position for a period of time. Some examples of time periods that may be used include about 10 seconds, about 15 seconds, and about 20 seconds. In one case, the time period may be in a range from about 10 seconds to about 20 seconds. The step size between positions may be defined by the elevational resolution of the 2d reconstructed image for the PACT implementation being used. For example, if the elevational resolution is 3-4 cm thick, the step size may be 3-4 cm. An example of a commercially-available mechanism for linear movement is a linear stage KR4610D made by THK America, Inc.

The one or more DAQ(s) 160 record photoacoustic signals at time intervals defined by a sampling frequency. In one example, the sampling frequency is in a range from about 4 MHz to about 100-Hz. In another example, the sampling frequency is 40 MHz.

According to one aspect, the one or more DAQs and one or more pre-amplifiers of a PACT system provide one-to-one mapped associations with the transducers in the ultrasonic transducer array. These one-to-one mapped associations allow for fully parallelized data acquisition of all ultrasonic transducer channels and avoids the need for multiplexing after each laser pulse excitation or other modulated or pulsed excitation illumination. With one-to-one mapped associations between pre-amplifiers and transducer elements, each ultrasound transducer element in the array is in electrical communication with one dedicated pre-amplifier channel (also referred to as “preamp channel”). The one dedicated pre-amplifier channel is configured to amplify only photoacoustic signals detected by the one associated/mapped ultrasound transducer. These one-to-one mapped associations between the transducers and the pre-amplifier channels allow for parallelized pre-amplification of the photoacoustic signals detected by the plurality of transducers in the ultrasound transducer array. With one-to-one mapped analog-to-digital sampling, each pre-amplifier is operatively coupled to a corresponding dedicated data channel of an analog-to-digital sampling device in a DAQ to enable parallelized analog-to-digital sampling of the plurality of pre-amplified PA signals. The pre-amplified PA signals produced by each individual preamp channel are received by a single dedicated data channel of the at least one analog-to-digital sampling devices. Any suitable number of pre-amplifier devices and/or DAQ devices may be used to provide the one-to-one mapping. For example, a PACT system may include four 128-channel DAQs (e.g., SonixDAQ made by Ultrasonix Medical ULC with 40 MHz sampling rate, 12-bit dynamic range, and programmable amplification up to 51 dB) in communication with four 128-channel pre-amplifiers to provide simultaneous one-to-one mapped associations with a 512-element transducer array. This PACT system can acquire photoacoustic signals from a cross section within 100 μs without multiplexing after each laser pulse excitation. The plurality of pre-amplifier channels may be directly coupled to the corresponding plurality of ultrasound transducers or may be coupled with electrical connecting cables. In one aspect, wireless communication may be employed.

Each of the one or more pre-amplifiers of a PACT system with spatiotemporal antialiasing may be set to a pre-amplifier gain that may be determined by one or more factors. For example, the pre-amplifier gain may be determined based on one or more of a minimum signal-to-noise ratio and one or more operating parameters of the data acquisition and processing system components such as analog-to-digital sampling devices (digitizers) of the DAQs, signal amplifiers, buffers, and the computing device. In one aspect, the pre-amplifier gain is in a range that is high enough to enable transmission of the photoacoustic signals with minimal signal contamination, but below a gain that may saturate the dynamic ranges of the DAQ system used to digitize the photoacoustic signals amplified by the pre-amplifier(s). In certain aspects, the gain of the plurality of pre-amplifier channels may be at least about 5 dB, at least about 7 dB, at least about 9 dB, at least about 11 dB, at least about 13 dB, at least about 15 dB, at least about 17 dB, at least about 19 dB, at least about 21 dB, at least about 23 dB, at least about 25 dB, or at least about 30 dB.

According to one aspect, a PACT system with spatiotemporal antialiasing includes a plurality of multi-channel preamplifiers (e.g., 128-channel preamplifiers) in electrical communication with an N-element ultrasonic transducer array and a plurality of multi-channel data acquisition systems (DAQs) in electrical communication with the plurality of pre-amplifiers respectively. Each of the DAQs is in communication with one of the preamplifiers. The plurality of multi-channel preamplifiers and the plurality of DAQs may also be in one-to-one mapping association with the N element ultrasonic transducer array. For example, a set of four 128-channel preamplifiers may be in electrical communication with a 512-element ultrasonic transducer array and a set of four 128-channel data acquisition systems (DAQs) may be in electrical communication with four 128-channel pre-amplifier(s) respectively. The four sets of 128-channel DAQs provide simultaneous one-to-one mapped associations with the 512-element ultrasonic transducer array to enable acquiring photoacoustic signals from a cross section without multiplexing after each laser pulse excitation.

Returning to FIG. 1, the PACT system with spatiotemporal antialiasing 100 also includes a computing device 180 having one or more processors or other circuitry 182, an optional (denoted by dashed line) display 186 in electrical communication with the processor(s) 182, and a computer readable media (CRM) 184 in electronic communication with the processor(s) or other circuitry 182. The computer readable media (CRM) 184 may be, e.g., a non-transitory computer readable media. Optionally (denoted by dashed line), the computing device 180 is in electronic communication with the light source(s) 110 to send control signals to trigger the illumination (e.g., triggering laser pulses). The computing device 180 is in electrical communication with the one or more DAQs 160 to receive data transmissions and/or to send control signal(s). Optionally (denoted by dashed line), the computing device 180 is in electronic communication with the one or more pre-amplifiers 150 to send control signal(s), e.g., to adjust amplification. The computing device 180 is in electrical communication with the antialiasing filter 181 to send control signals to adjust the cutoff frequency. The electrical communication between system components of the PACT system 100 may be in wired and/or wireless form. One or more of the electrical communications between components of the PACT system 100 may be able to provide power in addition to communicate signals. The computing device 180 may be, for example, a personal computer, an embedded computer, a single board computer (e.g. Raspberry Pi or similar), a portable computation device (e.g. tablet), a controller, or any other computation device or system of devices capable of performing the functions described herein. The computing device 180 is in electronic communication with the mechanism 170 to send control signals to control the movement and/or hold positions of the ultrasonic transducer array 140. The processor(s) 182 are in electrical communication with the CRM 184 to store and/or retrieve data such as the photoacoustic signal data. The one or more processor(s) and/or other circuitry 182 are in electrical communication with the optional display 186 to display data. Although not shown, the computing device 180 may also include a user input component for receiving data from a user.

The one or more processors and/or other circuitry 182 execute instructions stored on the CRM 184 to perform one or more operations of PACT methods. In certain implementations, the processor(s) 182 and/or other circuitry 182 execute instructions to perform one or more of: 1) determining an upper cutoff frequency for the antialiasing filter to trigger the antialiasing filter to comment temporal filtering; 2) communicating control signals to one or more components of the PACT system with spatiotemporal antialiasing 100, and 3) performing one or more reconstruction operations to reconstruct a two-dimensional or three-dimensional photoacoustic image of the specimen 130 using photoacoustic data. For example, the processor(s) and/or other circuitry 182 and/or one or more external processors may execute instructions that communicate control signals to the mechanism 170 to scan the ultrasonic transducer array 140 along a z-axis between to two elevations (3D mode) or move the ultrasonic transducer array 140 to one or more different elevations (2D mode) and send control signals to the digitizer in the DAQ(s) 160 to simultaneously record photoacoustic signals received by ultrasonic transducer array 140 from the illuminated regions of the specimen.

In some implementations, the PACT system includes one or more communication interfaces (e.g., a universal serial bus (USB) interface). Communication interfaces can be used, for example, to connect various peripherals and input/output (I/O) devices such as a wired keyboard or mouse or to connect a dongle for use in wirelessly connecting various wireless-enabled peripherals. Such additional interfaces also can include serial interfaces such as, for example, an interface to connect to a ribbon cable. It should also be appreciated that the various system components can be electrically coupled to communicate with various components over one or more of a variety of suitable interfaces and cables such as, for example, USB interfaces and cables, ribbon cables, Ethernet cables, among other suitable interfaces and cables.

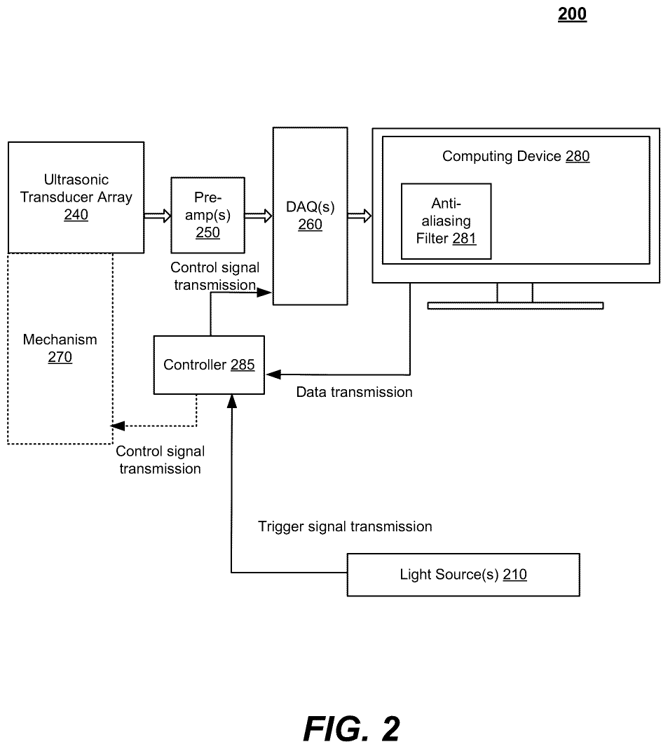

FIG. 2 is signal flow diagram of signals communicated between components of a PACT system with spatiotemporal antialiasing 200, according to implementations. The PACT system 200 includes one or more light sources 210 (e.g., a pulsed laser) and an optical system (not shown) that is configured to propagate/alter illumination from the one or more light sources 210 to a specimen being imaged. The PACT system 200 also includes an ultrasonic transducer array 240 that is coupled to or otherwise in acoustic communication with a specimen to receive photoacoustic signals induced by the illumination. The PACT system 200 also includes a optional (denoted by dotted line) mechanism 270 (e.g., a linear scanner) coupled to or otherwise operably connected to the ultrasonic transducer array 240 that can move the ultrasonic transducer array 240 to one or more elevational positions and/or scan the ultrasonic transducer array 240 between two elevational positions. The PACT system 200 also includes one or more pre-amplifiers 250 and one or more data acquisition systems (DAQs) 260 in one-to-one mapped association with the transducers in the ultrasonic transducer array 240. The one or more pre-amplifiers 250 are in electrical communication with the ultrasonic transducer array 240 to receive one or more photoacoustic signals and the DAQ(s) 260 are in electrical communication with the pre-amplifier(s) 250 to receive one or more boosted photoacoustic signals.

The PACT system with spatiotemporal antialiasing 200 also includes a computing device 280 having one or more processors or other circuitry and a computer readable media (CRM) in electronic communication with the processor(s). The PACT system 200 includes an anti-aliasing filter that may reside on the computer readable media. The antialiasing filter 281 is in electrical communication with the DAQ(s) 250 to receive photoacoustic data. The computing device 280 includes instructions residing on the computer readable media that can be executed to perform functions of the system 200 such as image reconstruction, spatial interpolation and determining an upper cutoff frequency of the anti-aliasing filter 281 and determining when to trigger temporal filtering.

The PACT system 200 also includes a controller 285 in electronic communication with the DAQ(s) 260 and the mechanism 270 to send control signals. The controller 285 may include one or more processors. To synchronize components of the PACT system 200, the one or more light sources 210 are configured to transmit a trigger signal to the controller 284 that triggers transmission of control signals to the DAQ(s) 260 to record signals and the mechanism 270 to move the ultrasonic transducer array 240. The computing device 280 is in communication with the controller 285 to be able to transmit control signals. The electrical communication between system components of the PACT system 200 may be in wired and/or wireless form. The electrical communications may be able to provide power in addition to communicate signals in some cases. During operation, digitized radio frequency data from the antialiasing filter 281 may be first stored in an onboard buffer, and then transferred to the computing device 280, e.g., through a universal serial bus 2.0. The DAQ(s) 260 may be configured to record photoacoustic signals within a time period, e.g., 100 μs, after each laser pulse excitation.

### Examples of PACT Methods with Spatiotemporal Antialiasing

The PACT methods described in this section can be used to obtain one or more two-dimensional images and/or one or more three-dimensional volumetric images. The operations of these PACT methods can be performed by one or more components of a the PACT system with spatiotemporal antialiasing such as, e.g., the PACT system with spatiotemporal antialiasing 100 shown in FIG. 1 or the PACT system with spatiotemporal antialiasing 200 shown in FIG. 2. One or more of the operations may be performed executing instructions retrieved from computer readable media.

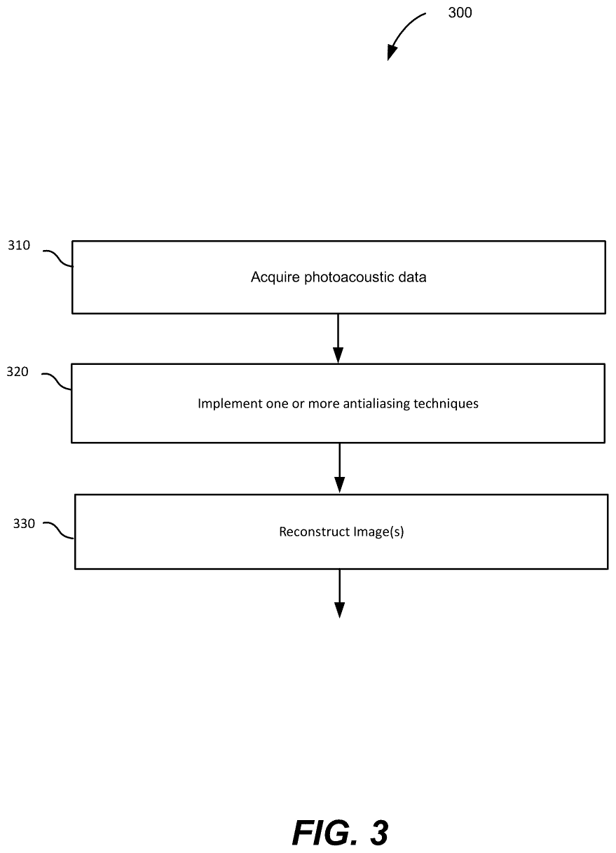

FIG. 3 is a flowchart depicting operations of a PACT method with spatiotemporal antialiasing that applies one or more antialiasing techniques (e.g., spatial interpolation and location-based temporal filtering), according to various implementations. For example, one implementation is directed to a method that applies both spatial interpolation and location-based temporal filtering, and then applies universal back-propagation during image reconstruction. Spatial interpolation may be applied after location-based on temporal filtering in this example.

At operation 310, photoacoustic data is acquired. For example, one or more control signals may be sent to component(s) of the PACT system to perform data acquisition operations or recorded photoacoustic data may be retrieved from a computer readable media such as an onboard buffer.

In one aspect, the PACT system may control operations of system components to perform data acquisition. During the data acquisition phase, the recording of photoacoustic data by the one or more data acquisition systems (DAQs) may be synchronized with light pulses from the one or more light sources. To synchronize data acquisition with light pulses from the light source(s), the external trigger from the light source(s) may be used to trigger recording by the data acquisition systems and/or trigger movement of any mechanism that may be moving the transducer array according to one aspect. For example, during data acquisition in an exemplary two-dimensional (2D) mode, the ultrasonic transducer array may be moved to one or more elevational positions (e.g., different locations z₁, z ₂, . . . along a z-axis of a linear stage) and held in each elevational position for a period of time during which the one or more DAQs record data. Some examples of time periods that may be used include: about 10 seconds, about 15 seconds, and about 20 seconds. In another example, the time period is in a range of about 10 seconds to about 20 seconds. At each cross section, photoacoustic data is continuously recorded at a certain sampling rate to monitor the cross section. As another example, during data acquisition in an exemplary three-dimensional (3D) mode, the ultrasonic transducer array is scanned through multiple scanning steps (elevational positions) through a depth.

During an exemplary data acquisition phase, digitized radio frequency data from the DAQs may be stored in an onboard buffer, and then transmitted to the computing device through e.g., a universal serial bus 2.0 or a universal serial bus 3.0. The photoacoustic data is recorded by the one or more data acquisition systems at a sampling frequency. In one aspect, the sampling frequency is 40 MHz. In another aspect, the sampling frequency may be in a range from 4 MHz to 100 MHz. The one or more data acquisition systems may be set to record photoacoustic data within a particular time period (e.g., 100 μs, 200 μs, or 300 μs) after each illumination e.g., laser pulse excitation. In certain implementations, a PACT system with spatiotemporal antialiasing is equipped with a one-to-one mapped signal amplification and data acquisition (DAQ) systems or DAQ circuits to the transducer elements. If there is one-to-one mapping associations, the one or more data acquisition systems can acquire photoacoustic signals from a cross-section of a specimen without multiplexing after illumination e.g., after each laser pulse excitation.

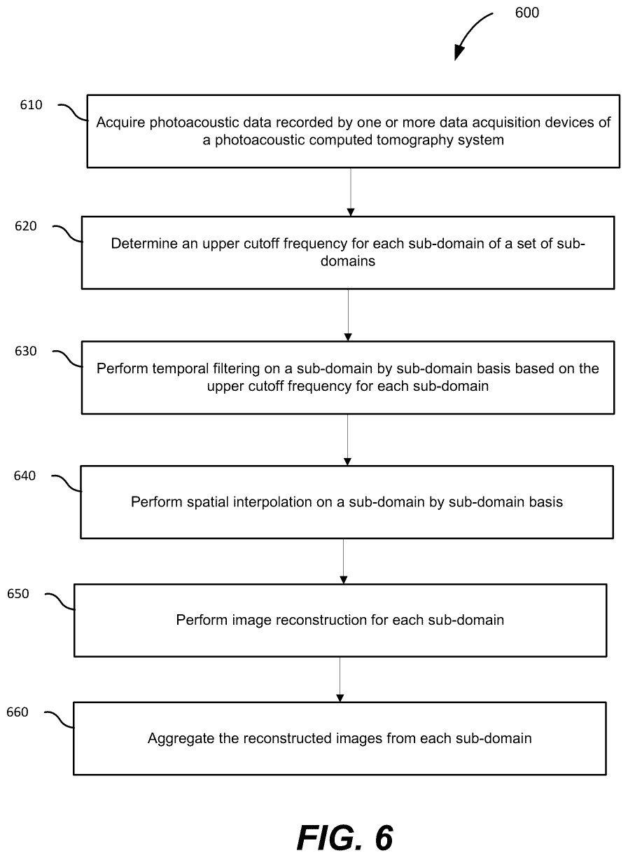

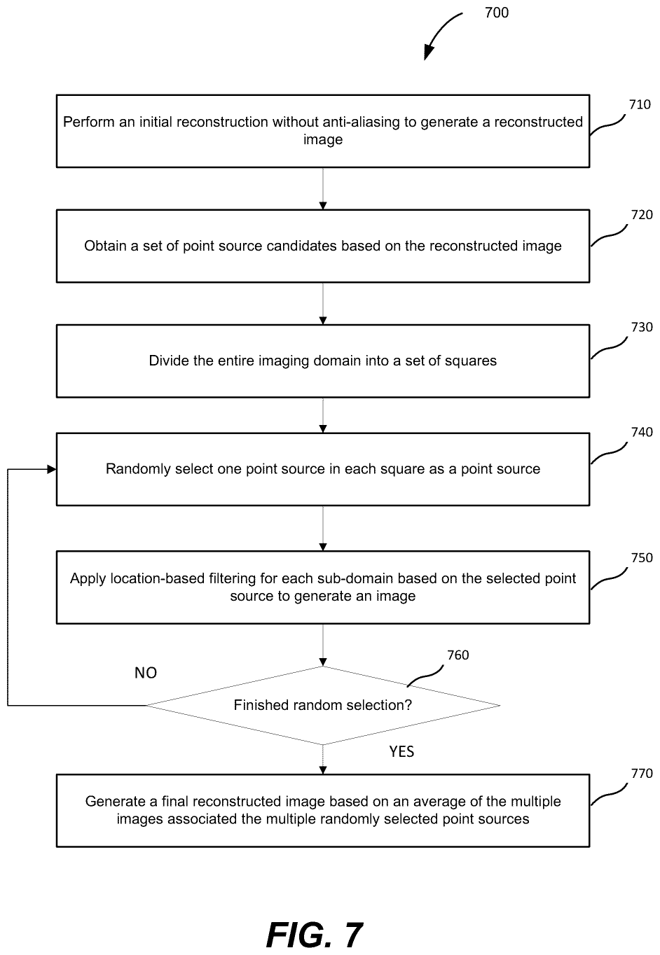

Returning to FIG. 3, at operation 320, one or more antialiasing techniques are implemented. For example, one or both of (i) location-based temporal filtering, and (ii) spatial interpolation may be implemented. If both spatial interpolation and location-based temporal filtering are implemented, location-dependent temporal filtering is applied before spatial interpolation according to one aspect. The location-based temporal filtering and/or the spatial interpolation may be performed on a sub-domain by sub-domain basis, as shown in and described below in connection with FIGS. 6 and 7. In some implementations, one or both of (i) location-based temporal filtering, and (ii) spatial interpolation may be used with UBP reconstruction.

At operation 330, the PACT system performs image reconstruction. Image reconstruction may include (i) reconstructing one or more two-dimensional images at different positions of the ultrasonic transducer array and/or (ii) reconstructing a volumetric three-dimensional image for a depth scanned by the ultrasonic transducer array. Image reconstruction includes, at least in part, implementing an inverse reconstruction algorithm. Some examples of inverse reconstruction algorithms that can be used include: (i) forward-model-based iterative methods, (ii) time-reversal methods, and (iii) universal back-projection (UBP) method. For example, a 3D back projection algorithm can be used to reconstruct a 3D volumetric image or a 2D back projection algorithm can be used to reconstruct a 2D image. An example of a universal back-projection process can be found in Xu, M. And Wang, L., “Universal back-projection algorithm for photoacoustic computed tomography,” Physical Review E 71, 016706 (2005), which is hereby incorporated by reference in its entirety. Another example of a back-projection process can be found in Anastasio, M. A. et al., “Half-time image reconstruction in thermoacoustic tomography,” IEEE Trans., Med. Imaging 24, pp 199-210 (2005), which is hereby incorporated by reference in its entirety. In another aspect, a dual-speed-of sound (dual-SOS) photoacoustic reconstruction process may be used. Additional examples of inverse reconstruction algorithms are provided in Section I. An example of a single-impulse panoramic photoacoustic computed tomography system that employs a dual-SOS photoacoustic reconstruction process is described in U.S. patent application 2019/0307334, titled “SINGLE-IMPULSE PANORAMIC PHOTOACOUSTIC COMPUTED TOMOGRAPHY” and filed on May 29, 2019, which is hereby incorporated by reference in its entirety.

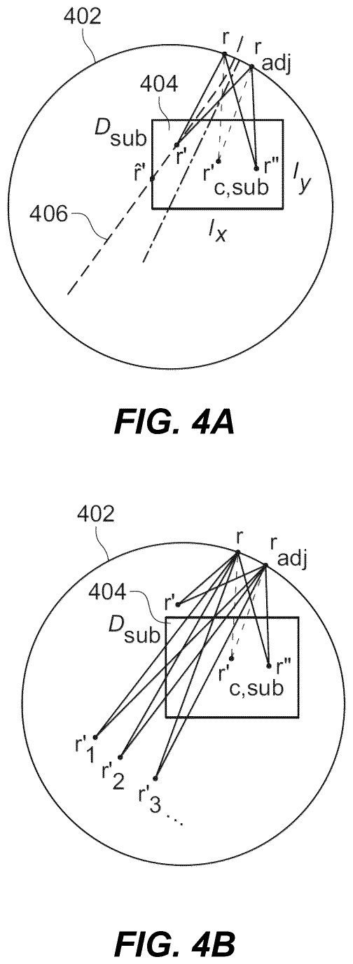

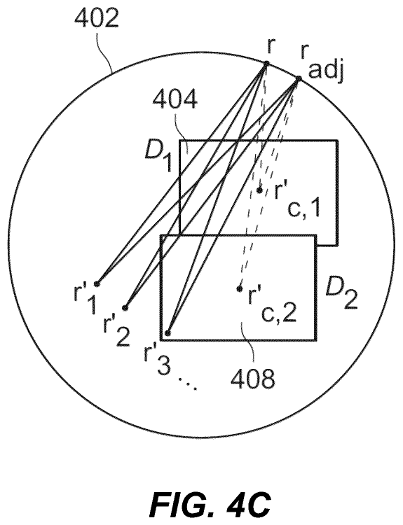

FIG. 4 illustrates examples of imaging sub-domains with sources inside and outside a given sub-domain. In the example illustrated in diagram (a) of FIG. 4, there are no point sources outside the depicted imaging sub-domain. In particular, a full-ring transducer array 402 and an imaging sub-domain 404 (represented by D_{sub}) are illustrated. Imaging sub-domain 404 is centered at r_{c,sub}′ and has a size of lₓ×l_{y}. Two adjacent transducer elements, r, and r_{adj} are shown. There is a point source r′ within imaging sub-domain 404, and a reconstruction location r″ inside of imaging sub-domain 404. A hyperbola exists with points r and r_{adj} as foci, with one branch 406 crossing point source r′. An intersection point of branch 406 with the boundary of imaging sub-domain 404 is denoted as \hat{r}′. In some embodiments, as described in more detail below in connection with FIG. 6, an upper cutoff frequency for location-based temporal filtering may be selected such that the Nyquist criterion is satisfied for each sub-domain. In particular, in some embodiments, the upper cutoff frequency may be determined based on the relative locations of the transducer elements, the center of the sub-domain, and points on the boundary of the imaging sub-domain.

In the example illustrated in diagram (b) of FIG. 4, there are one or multiple point sources outside of imaging sub-domain 404, with a reconstruction location r″ inside of imaging sub-domain 404. Diagram (b) illustrates multiple point sources r′, r₁′, r₂′, and r₃′ that are outside imaging sub-domain 404. In some embodiments, an upper cutoff frequency for location-based temporal filtering may be selected such that the Nyquist criterion is satisfied for each sub-domain. In particular, in some embodiments, the upper cutoff frequency may be determined based on the relative locations of the transducer elements, the center of the imaging sub-domain, the and the location of the point source.

In the example illustrated in diagram (c) of FIG. 4, there are multiple point sources outside of multiple imaging sub-domains 404 and 408, centered at r_{c,1}′ and r_{c,2}′, respectively. The multiple point sources are represented as r₁′, r₂′, and r₃′.

FIG. 5 is a flowchart of an example process 500 for performing spatiotemporal filtering on photoacoustic data in accordance with some embodiments. Blocks of process 500 may be performed by a computing device, such as a computing device associated with a PACT system, a server device, or the like. In some implementations, blocks of process 500 may be executed in an order other than what is shown in FIG. 5. In some embodiments, two or more blocks of process 500 may be executed substantially in parallel. In some embodiments, one or more blocks of process 500 may be omitted.

Process 500 can begin at 510 by acquiring photoacoustic data recorded by one or more data acquisition device of a photoacoustic computed tomography system. An example of a photoacoustic computed tomography system is shown in and described above in connection with FIGS. 1 and 2.

At 520, process 500 can apply location-based temporal filtering to the photoacoustic data acquired. Applying the location-based temporal filtering may be on a sub-domain by sub-domain basis for a set of sub-domains, where the set of sub-domains, when aggregated, form an entire imaging domain. In some embodiments, applying the location-based temporal filtering may involve determining an upper cutoff frequency for each sub-domain and applying one or more lowpass filters to the photoacoustic data on a sub-domain by sub-domain basis using the upper cutoff frequency for the corresponding sub-domain. By determining the upper cutoff frequency for each sub-domain, the Nyquist criterion may be satisfied for each sub-domain. In instances where a point source is within a given sub-domain, the upper cutoff frequency may be determined based on relative locations of the ultrasound transducer elements, a center of the sub-domain, points on the boundary of the sub-domain. In instances where a point source is outside of a given sub-domain, the upper cutoff frequency may be determined based on relative locations of the ultrasound transducer elements, a center of the sub-domain, and the point source. Note that more detailed example techniques for determining the upper cutoff frequency are described below in connection with FIG. 6.

In instances in which a general source (rather than a point source) causes reflections indicated in the photoacoustic data, applying the location-based temporal filtering may involve obtaining a set of point source candidates based on an initial reconstruction of the photoacoustic data, dividing the entire imaging domain into a set of rectangles, such as a set of squares, randomly selecting a point source in each rectangle as a point source, and applying the location-based filtering for each sub-domain based on the selected point source (e.g., as described above and/or as described below in connection with FIG. 6). The random selection of point sources may be repeated, and the results of the filtering for each randomly selected point source may be aggregated when generating the final reconstructed image. More detailed example techniques for applying location-based temporal filtering in the case of a general source are shown in and described below in connection with FIG. 7. Note that, as used herein, a “point source” refers to a point with a diameter smaller (e.g., substantially smaller) than the resolution of the system. A “general source” may have no limitation on the form of the source, which may be a point, or a continuous distribution. By way of analogy, a “point source” versus a “general source” may be thought of similar to a dot versus a full picture.

In some embodiments, after performing location-based temporal filtering, spatial interpolation may be performed. The spatial interpolation may be performed on a sub-domain by sub-domain basis. Note that, the location-based temporal filtering may mitigate aliasing prior to spatial interpolation, thereby allowing spatial resolution to be preserved while mitigating aliasing.

At 530, process 500 can reconstruct one or more photoacoustic images from the filtered photoacoustic data. In instances in which spatial interpolation was performed, the reconstructed images may be reconstructed based on the filtered and interpolated data. In some implementations, the one or more photoacoustic images may be reconstructed using universal back projection.

FIG. 6 is a flowchart of an example process 600 for performing spatiotemporal filtering on photoacoustic data in accordance with some embodiments. Note that blocks of process 600 may be performed for point sources rather than general sources which cause reflections in acquired photoacoustic data. In some implementations, in an instance of a general source, corresponding point sources may be obtained (e.g., using the techniques shown in and described below in connection with FIG. 7), and location-based temporal filtering and spatial interpolation may then be performed using the obtained point sources and the techniques described in process 600. In some embodiments, blocks of process 600 may be performed by one or more computing devices, such as a computing device associated with a PACT system, a server device, or the like. An example of such a PACT system is shown in and described above in connection with FIG. 1. In some implementations, blocks of process 600 may be executed in an order other than what is shown in FIG. 6. In some embodiments, two or more blocks of process 600 may be executed in an order other than what is shown in FIG. 6. In some embodiments, one or more blocks of process 600 may be omitted.

Process 600 can begin at 610 by acquiring photoacoustic data recorded by one or more data acquisition device of a photoacoustic computed tomography system. An example of a photoacoustic computed tomography system is shown in and described above in connection with FIGS. 1 and 2.

At 620, process 600 can determine an upper cutoff frequency for sub-domain of a set of sub-domains. A given sub-domain is generally represented herein as D su b. The upper cutoff frequency for a given sub-domain may be one that satisfies the Nyquist criterion for that sub-domain. Note that, in instances in which the upper cutoff frequency is being determined for a point source within a sub-domain, the upper cutoff frequency may be determined based on the relative locations of: the transducer elements; the center of the sub-domain; and points on the boundary of the sub-domain. In instances in which the upper cutoff frequency is being determined for a point source outside of a sub-domain, the upper cutoff frequency may be determined based on the relative locations of: the transducer elements; the center of the sub-domain, and the location of the point source.

For a source point r′ within the sub-domain, and a reconstruction location r″ within the sub-domain, spatial aliasing in spatial sampling may be analyzed. Given two adjacent transducer element locations r and r_{adj}, the parameter tᵣ′ may be defined by:

$${t}_{r}^{′} = \frac{‖ {r}^{′} - r ‖}{c} - \frac{‖ {r}_{c , sub}^{′} - r ‖}{c} \qquad ( Eqn . 11 )$$

The step size when element location r changes to r_{adj} may be determined by:

$$τ ( r , {r}_{adj} , {r}_{c , sub}^{′} , {r}^{′} ) = \vert {t}_{radj}^{′} - {t}_{r}^{′} \vert = \vert ( \frac{‖ {r}^{′} - {r}_{adj} ‖}{c} - \frac{‖ {r}^{′} - r ‖}{c} ) - ( \frac{‖ {r}_{c , sub}^{′} - {r}_{adj} ‖}{c} - \frac{‖ {r}_{c , sub}^{′} - r ‖}{c} ) \vert \qquad ( Eqn . 12 )$$

For any r′ within the sub-domain D_{sub}, with ∥r′−r∥≠∥r′−r_{adj}∥, there exists a branch of a hyperbola crossing r′ with r and r_{adj} as the foci. Either one of the intersection points between the branch and the boundary of D_{sub} is represented herein by \hat{r}′. The boundary of the sub-domain D_{sub} is generally represented herein as ∂D_{sub}. Based on the definition of the hyperbola,

$$\frac{‖ {r}^{′} - {r}_{adj} ‖}{c} - \frac{‖ {r}^{′} - r ‖}{c} = \frac{‖ {\hat{r}}^{′} - {r}_{adj} ‖}{c} - \frac{‖ {\hat{r}}^{′} - {r}_{adj} ‖}{c} .$$

For any r′ϵD_{sub} with ∥r′−r∥=∥r′−r_{adj}∥, r′ will be on the perpendicular bisector of the line segment with r and r_{adj} as endpoints. The step size associated with the sub-domain may be defined by:

$${τ}_{Dsub} ( r , {r}_{adj} ) = {max}_{{r}^{′} ϵ \partial Dsub} τ ( r , {r}_{adj} , {r}_{c , sub}^{′} , {r}^{′} ) \qquad ( Eqn . 13 )$$

For any r′ϵD_{sub}, replacing r′ with \hat{r}′ in τ(r, r_{adj}, r_{c,sub}′, r′) does not change its value, yielding:

τ(r,r_{adj},r_{c,sub}′,r′)=τ(r,r_{adj},r_{c,sub}′,\hat{r}′)≤τ_{Dsub}(r,r_{adj}) (Eqn. 14)

The computational complexity of estimating the upper limit of the step size τ(r, r_{adj}, r_{c,sub}′, r′) may be reduced from searching r′ in D_{sub} to searching r′ on the boundary of the sub-domain, i.e., searching r′ on ∂D_{sub}. By searching on the boundary of the sub-domain, the computational complexity may be reduced by one dimension. The upper cutoff frequency f_{c,SS} for spatial sampling must meet the Nyquist criterion specified by:

$${f}_{c , SS} < \frac{1}{2 {τ}_{Dsub} ( r , {r}_{adj} )} \qquad ( Eqn . 15 )$$

For the sub-domain D_{sub} and an element rₙ, the upper cutoff frequency may be determined by:

$${f}_{c , Dsub} ( {r}_{n} ) = {min}_{{r}_{n ′} is adjacent to {r}_{n}} \frac{1}{2 {τ}_{Dsub} ( {r}_{n} , {r}_{n}^{′} )} , n , {n}^{′} = 1 , 2 , … N \qquad ( Eqn . 16 )$$

For a point source r′ outside the sub-domain, the upper cutoff frequency for spatial sampling for a given sub-domain D_{sub} may be determined by:

$${f}_{c , SS , Dsub , OS} ( {r}_{n} , {r}^{′} ) = {min}_{{r}_{n ′} is adjacent to {r}_{n}} \frac{1}{2 τ ( {r}_{n} , {r}_{{n}^{′}} , {r}_{c , sub}^{′} , {r}^{′} )} , n , {n}^{′} = 1 , 2 , … N \qquad ( Eqn . 17 )$$

In some cases, there may be signals from both the sub-domain D_{sub} and the source point r′. In these cases, the upper cutoff frequency may be presented for the time domain subsets as:

$${f}_{c , SS , Dsub , OS} ( {r}_{n} , {t}^{′} , {r}^{′} ) = \{ \begin{matrix}{f}_{c , SS , Dsub , OS} ( {r}_{n} , {r}^{′} ) , & {t}^{′} - {t}_{0}^{′} ϵ {T}_{e} \\ {f}_{c , IS} , & else\end{matrix} , n = 1 , 2 , … , N \qquad ( Eqn . 18 )$$

In the equation above,

$${t}_{0}^{′} = \frac{‖ {r}^{′} - {r}_{n} ‖}{c} - \frac{‖ {r}_{c , sub}^{′} - {r}_{n} ‖}{c}$$

denotes the recentered first arrival time from r′ to rₙ, and f_{c,IS} denotes the upper cutoff frequency of the imaging system. The upper cutoff frequency for the recentered signal \hat{p}_{Dsub} (rₙ, t′) may be determined by:

f_{c,Dsub,r′}(rₙ,t′)=min{f_{c,Dsub}(rₙ),f_{c,SS,Dsub,OS}(rₙ,t′,r′)},n=1,2, . . . N (Eqn. 19)

For the n^{th} element at time t′, by applying lowpass filtering with the above upper cutoff frequency, aliasing in spatial sampling may be removed for signals from both the subdomain D_{sub} and the point source r′.

In the case of multiple point sources outside of the sub-domain, where the set of point sources are represented as G={r₁′,r₂′, . . . }, the upper cutoff frequency may be determined by:

$${f}_{c , Dsub , G} ( {r}_{n} , {t}^{′} ) = min \{ {f}_{c , Dsub} ( {r}_{n} ) , {min}_{r ′ϵ G} {f}_{c , SS , Dsub , OS} ( {r}_{n} , {t}^{′} , {r}^{′} ) \} , n = 1 , 2 , … , N \qquad ( Eqn . 20 )$$

To remove spatial aliasing in spatial sampling for signals in the sub-domain D_{sub} for the point sources in G, lowpass filtering may be applied with the upper cutoff frequency given above to the recentered signal \hat{p}_{Dsub}(rₙ, t′) of the n^{th} element at time t′.

At 630, process 600 may perform temporal filtering on a sub-domain by sub-domain basis based on the upper cutoff frequency for each sub-domain. For example, in some embodiments, a filter with the upper cutoff frequency may be implemented using a lowpass filter (e.g., a Butterworth filter). In one example, the lowpass filter may be a third-order Butterworth filter. The lowpass filter may be combined with a sinc filter with the same upper cutoff frequency.

Note that, in the instance in which the point source r′ is outside the sub-domain, to remove aliasing in spatial sampling for signals from the point source r′ that is outside the sub-domain (denoted by “OS” in the equation above), lowpass filtering may be applied to \hat{p}_{Dsub}(rₙ, t) using the upper cutoff frequency determined from the equation above. To minimize unwanted smoothing of signals, \hat{p}_{Dsub}(rₙ, t) may be filtered only for

$${t}^{′} - ( \frac{‖ {r}^{′} - {r}_{n} ‖}{c} - \frac{‖ {r}_{c , sub}^{′} - {r}_{n} ‖}{c} ) ϵ [ 0 , {T}_{e} ] .$$

Tₑ refers to the non-zero duration of hₑ′(t), meaning that hₑ′(t) is only non-zero on [0, Tₑ].

At 640, process 600 can perform spatial interpolation on a sub-domain by sub-domain basis. Note that, as discussed above, after performing temporal filtering, the Nyquist criteria are satisfied with respect to the detected photoacoustic signals. Using the temporally filtered signal, the signal may be recovered numerically, and spatial interpolation may then be performed on the recovered signal to fully remove aliasing. Spatial interpolation may remove aliasing caused by the reconstruction.

Spatial interpolation may be performed by determining a step size associated with interpolated transducer elements that satisfy the Nyquist criteria for reconstruction. In other words, given N physical transducer elements, spatial interpolation may generate an effective number of transducer elements that includes intermediate virtual transducer elements, where the effective number of transducer elements corresponds to the number of physical transducer elements N multiplied by a factor of β. Process 600 may determine β subject to the Nyquist criteria on a sub-domain by sub-domain basis.

In some embodiments, spatial interpolation may involve analyzing spatial aliasing in the image reconstruction. For example, the upper limit of the step size of

$$\frac{‖ {r}^{″} - {r}_{adj} ‖}{c} - \frac{‖ {r}^{″} - r ‖}{c}$$

between two adjacent transducer element locations, r and r_{adj}, may be estimated. In some embodiments, the upper limit of the step size may be estimated by:

$$τ ( r , {r}_{adj} , {r}^{′} , {r}^{″} ) = \vert ( \frac{‖ {r}^{″} - {r}_{adj} ‖}{c} - \frac{‖ {r}^{″} - r ‖}{c} ) - ( \frac{‖ {r}^{′} - {r}_{adj} ‖}{c} - \frac{‖ {r}^{′} - r ‖}{c} ) \vert , \qquad ( Eqn . 21 )$$

$${r}^{′} , {r}^{″} ϵ {D}_{sub}$$

For point sources within the sub-domain, the triangle inequality may be used to obtain:

τ(r,r_{adj},r′,r″)≤τ(r,r_{adj},r_{c,sub}′,r′)+τ(r,r_{adj},r_{c,sub}′,r″)≤2τD_{sub}(r,r_{adj}),r′,r″ϵD_{sub} (Eqn. 22)

Based on the above, it may be determined that aliasing may be removed in the image reconstruction by performing additional lowpass filtering with an upper cutoff frequency of

$$\frac{{f}_{c , Dsub ( {r}_{n} )}}{2} , n = 1 , 2 , … , N .$$

Note that this upper cutoff frequency is half the value required for removing aliasing in the spatial sampling. The additional lowpass filtering may be omitted if aliasing in the spatial sampling is removed prior to spatial interpolation. The denser transducer element locations after the spatial interpolation may be denoted as r_{β,n}=1, 2, . . . , βN, where β is an integer. Note that the denser element locations coincide with the physical element locations at rₙ=r_{β,β(n-1)+1}, n=1, 2, . . . , N. For each t′, the recentered signals (represented herein as \hat{p}_{Dsub}(rₙ, t′)), from all transducer elements form a vector of length N. A fast Fourier transform (FFT) may be applied to the vector, and zeros may be padded following the highest frequency components to form a new vector of length βN. An inverse FFT may be applied to the new vector to complete the spatial interpolation. The cutoff frequency may be updated based on the denser transducer element locations as:

$${f}_{c , Dsub , β} ( {r}_{β , n} ) = {min}_{{r}_{β , {n}^{′} is adjacent to {r}_{β , n}}} \frac{1}{2 {τ}_{Dsub} ( {r}_{β , n} , {r}_{β , {n}^{′}} )} , \qquad ( Eqn . 23 )$$

$$n , {n}^{′} = , 1 , 2 , … , β N$$

Note that, the larger the value of β, the closer the adjacent transducer elements, the smaller the value of τ_{Dsub}(r_{β,n}, r_{β,n′}), and the larger the value of f_{c,Dsub,β}(r_{β,n}) The signals of the n^{th} channel may be filtered with the upper cutoff frequency

$$\frac{{f}_{c , Dsub , β} ( {r}_{β , n} )}{2}$$

to remove aliasing in the image reconstruction. To avoid compromising spatial resolution, β may be selected to satisfy the following criteria:

$$\frac{{f}_{c , Dsub , β} ( {r}_{β , β ( n - 1 ) + 1} )}{2} \geq {f}_{c , Dsub} ( {r}_{n} ) , n = 1 , 2 , … , N \qquad ( Eqn . 24 )$$

Therefore, after first performing temporal filtering with the upper cutoff frequency of f_{c,Dsub}(rₙ) to remove aliasing in the spatial sampling and the spatial interpolation with factor β, additional temporal filtering may be omitted. Because a general sub-domain is off-centered in the image domain, the spatial interpolation is applied to the recentered signals \hat{p}_{Dsub}(rₙ, t′) instead of the original signals \hat{p}(rₙ, t). In some embodiments, the minimum value of β that satisfies the above criteria may be obtained through numerical computations on a sub-domain by sub-domain basis. In one example, β may be 2.

For point sources outside the sub-domain, the triangle inequality may be used to obtain:

τ(r,r_{adj},r′,r″)≤τ(r,r_{adj},r′,r_{c,sub}′)+τ(r,r_{adj},r_{c,sub}′,r″)≤τ(r,r_{adj},r′,r_{c,sub}′)+τ_{Dsub}(r,r_{adj}) for r′ϵD\D_{sub} and r″ϵD_{sub} (Eqn. 25)

For the point source outside the sub-domain, the cutoff frequency for denser transducer element locations may be determined by:

$$\begin{matrix}{f}_{c , IR , Dsub , O S , β} ( {r}_{β , n} , {r}^{′} ) & = {min}_{{r}_{β , {n}^{′}} is adjacent to {r}_{β , n}} \frac{1}{\begin{matrix}( 2 ( τ ( {r}_{β , n} , {r}_{β , {n}^{′}} , {r}^{′} , {r}_{c , sub}^{′} ) + \\ {τ}_{Dsub} ( {r}_{β , n} , {r}_{β , {n}^{′}} ) )\end{matrix}} , n , {n}^{′} \\ & = 1 , 2 , … , β N\end{matrix} \qquad ( Eqn . 26 )$$

For the point source outside the sub-domain, a minimal β may be chosen to satisfy the following criteria:

f_{c,IR,Dsub,OS,β}(r_{β,β(n-1)+1},r′)≥f_{c,SS,Dsub,OS}(rₙ,r′),n=1,2, . . . ,N (Eqn. 27)

Similar to what is described above for point sources within the sub-domain, the value of β may be determined using numerical computations.

Temporal recentering may be reversed based on the relation:

$$\tilde{p} ( {r}_{β , n} , t ) = {\tilde{p}}_{Dsub} ( {r}_{β , n} , t - \frac{‖ {r}_{c , sub}^{′} - {r}_{β , n} ‖}{c} ) , n = 1 , 2 , … , β N , t \geq 0 \qquad ( Eqn . 28 )$$

At 650, process 600 may perform image reconstruction for each sub-domain. For example, the reconstruction may be performed based on:

$${\hat{p}}_{0} ( {r}^{″} ) \approx {\sum }_{n = 1}^{N} {ω}_{n} \hat{b} ( {r}_{n} , t = \frac{‖ {r}^{″} - {r}_{n} ‖}{c} ) , {r}^{″} ϵ {D}_{sub} \qquad ( Eqn . 29 )$$

At 660, process 600 may aggregate the reconstructed images for each sub-domain. For example, in some embodiments, the sub-domain images may be aggregated to form the whole image using:

$${\hat{p}}_{0} ( {r}^{″} ) \approx {\sum }_{i = 1}^{I} {\hat{ω}}_{lx ( Di ) , ly ( Di ) , ζ DD} ( {r}^{″} - {r}_{c , i}^{′} ) {\hat{p}}_{0 , Di} ( {r}^{″} ) , {r}^{″} ϵ D \qquad ( Eqn . 30 )$$

In the equation given above, the whole image domain is represented by D, and the multiple sub-domains are represented by D₁, D₂, . . . , Dₜ such that D=D₁ U D₂ U . . . U Dₜ. Adjacent sub-domains are overlapped by a length of C_{DD}.

Note that, in some embodiments, location-dependent temporal filtering may be implemented in a computationally efficient manner by utilizing precomputation and interpolation for filtering associated with the upper cutoff frequency. In particular, in some embodiments, rather than filtering the obtained photoacoustic signal for a given sub-domain for each element at time t′ (e.g., the photoacoustic signal represented as \hat{p}_{Dsub}(rₙ, t′)), the originally obtained signals \hat{p}(rₙ, t) at each transducer element may be filtered using a set of lowpass filters, each filter having a cutoff frequency less than the determined upper cutoff frequency f_{c}. In some embodiments, the set of filters may evenly (e.g., linearly) span the range of frequencies from 0 to f_{c}. For example, a set of k lowpass filters may have upper cutoff frequencies of f_{c,k}=1, 2, . . . , K+1 satisfying 0<f_{c,1}<f_{c,2}< . . . <f_{c,K}<f_{c,K+1}=f_{c}. Note that, in some embodiments, the set of cutoff frequencies associated with the set of lowpass filters may be dense enough such that further increasing the density has only minor effects on the reconstructed images. In some implementations, a lowpass filter having a given upper cutoff frequency may be a lowpass filter with the given upper cutoff frequency followed by a sinc filter with the same upper cutoff frequency. In one example, the lowpass filter may be a third-order Butterworth filter, although other filter implementations may be used.

The filtered signals may be represented as: \hat{p}f_{c,k}(rₙ, t), k=1, 2, . . . , K+1. For reconstruction associated with sub-domain D_{sub}, the filtered signals may be recentered, yielding:

$${\hat{p}}_{Dsub , {f}_{c , k}} ( {r}_{n} , {t}^{′} ) = {\hat{p}}_{{f}_{c , k}} ( {r}_{n} , {t}^{′} + \frac{‖ {r}_{c , Sub}^{′} - {r}_{n} ‖}{c} ) , \qquad ( Eqn . 31 )$$

$$n = 1 , 2 , … , N , k = 1 , 2 , … , K + 1$$

The filtered signals may then be used for spatial interpolation and reconstruction, as shown in and described above in connection with FIG. 6.

The process shown in and described above in connection with FIG. 6 relates to spatiotemporal filtering for point sources, whether inside or outside a given sub-domain. In instances in which spatiotemporal filtering is performed for a general source rather than a point source, a process for performing spatiotemporal filtering may involve performing an initial reconstruction without anti-aliasing, identifying a set of point source candidates based on the initial reconstruction, and applying location-based filtering multiple times for each sub-domain of a set of sub-domains using a randomly selected point source of the set of point sources. The location-based filtering may be performed, for each randomly selected point source, using, e.g., any of the techniques described above in connection with FIG. 6. Each application of location-based filtering may result in an image, and the final reconstructed image may be generated by averaging the multiple images generated by the multiple applications of location-based temporal filtering.

FIG. 7 is a flowchart of an example process 700 for spatiotemporal filtering for images involving general sources. In some embodiments, blocks of process 700 may be performed by one or more computing devices, such as a computing device associated with a PACT system, a server device, or the like. An example of such a PACT system is shown in and described above in connection with FIG. 1. In some implementations, blocks of process 700 may be executed in an order other than what is shown in FIG. 7. In some embodiments, two or more blocks of process 700 may be executed in an order other than what is shown in FIG. 7. In some embodiments, one or more blocks of process 700 may be omitted.

Process 700 may begin at 710 by performing an initial reconstruction on photoacoustic data, where the initial reconstruction does not utilize anti-aliasing. The result of the initial reconstruction is an initial reconstructed image. The initial reconstruction may be performed using universal backprojection. The initial reconstruction may be with respect to the entire imaging domain (e.g., rather than on a sub-domain by sub-domain basis). The initial reconstructed image may be represented herein as \hat{p}_{0,UBP}(r″),r″ϵD.

At 720, process 700 may obtain a set of point source candidates based on the initial reconstructed image. For example, in some embodiments, the set of point source candidates may be obtained by applying a threshold to the reconstructed image. As a more particular example, in some embodiments, a set of pixels of the reconstructed image having absolute values greater than a threshold may be considered point source candidates. In one example, given M pixels in the initial reconstructed image, and a candidacy ratio represented by α, the αM pixels with the largest absolute values may be selected as point source candidates.

At 730, process 700 can divide the entire imaging domain D into a set of squares. Each square may correspond to a sub-domain. Each square may have a size generally represented herein as I_{SP}×I_{SP}. Note that, although the imaging domain is generally described herein as divided into a set of squares, in some embodiments, the imaging domain may be divided into rectangles. Each rectangle may have a height and width that are different or the same (e.g., a square) as each other.

At 740, process 700 may, if one exists, randomly select one point source from each square as a selected point source from the set of point source candidates. Note that, for a given square of the set of squares, if no point source exists within the square, process 700 may omit selection of a point source for the square.

At 750, process 700 may apply location-based temporal filtering for the sub-domain corresponding to each square based on the selected point source to generate an image for the sub-domain. Location-based temporal filtering may be performed using, e.g., any of the techniques shown in and described above in connection with FIG. 6. In some embodiments, process 700 may apply location-based spatial interpolation, e.g., using any of the techniques shown in and described above in connection with FIG. 6.

At 760, process 700 can determine whether random selection of point source candidates has finished. If, at 760, process 700 determines that random selection of point source candidates has not finished (“no” at 760), process 700 can loop back to 740 and randomly select another point source in each square for which a point source candidate exists. Process 700 may loop through blocks 740-760 J times to form J groups of point sources, represented herein as G₁, G₂, . . . , G_{J}. J may be a value between, e.g., 1 and 200. For example, in some embodiments, J may be 1, 10, 50, 100, 200, or the like.

Conversely, if, at 760, process 700 determines that random selection of point source candidates has finished (“yes” at 760), process 700 can proceed to block 770 and can generate a final reconstructed image based on an average of the multiple images associated with images generated from the temporal filtering corresponding to the multiple randomly selected point sources. For example, the multiple images may be added and divided by the number of images to generate the average. To mosaic images from each sub-domain, represented as D₁, D₂, . . . Dₜ, a two-dimensional weight function may be defined as:

[symbol]

(r″)= [symbol] (x,y)= [symbol] (x) [symbol] (y) (Eqn. 32)

In the equation given above, C relates to an extension of the boundaries of a given sub-domain. For example, each sub-domain may be extended outside of its boundaries by

$$\frac{{DD}_{}}{2} .$$

A one-dimensional weight function may be defined as:

$${ω}_{l , ζ} ( x ) = \{ \begin{matrix}1 , & \vert x \vert \leq \frac{l}{2} \\ 1 - \frac{2 \vert x \vert - l}{} , & \frac{l}{2} < \vert x \vert \leq \frac{l +}{2} \\ 0 , & \vert x \vert > \frac{l +}{2}\end{matrix} \qquad ( Eqn . 33 )$$

The weight functions may be normalized for the sub-domains using:

$$( {r}^{″} - {r}_{c , i}^{′} ) = \frac{{ω}_{lx ( Di ) , ly ( Di ) , ζ DD} ( {r}^{″} - {r}_{c , i}^{′} )}{{\sum }_{{i}^{′} = 1}^{I} ( {r}^{″} - {r}_{c , {i}^{′}}^{′} )} , \qquad ( Eqn . 34 )$$

$$i = 1 , 2 , … . I$$

In the equation given above, lₓ(Dᵢ) and l_{y}(Dᵢ) represent the sizes of the rectangle Dᵢ in the x-axis and y-axis directions, respectively, and r_{c,i}′ represents the center of Dᵢ. The images in subdomains D₁, D₂, . . . Dₗ may be mosaiced to form the whole reconstructed image over the entire imaging domain D.

### Example Experimental Data

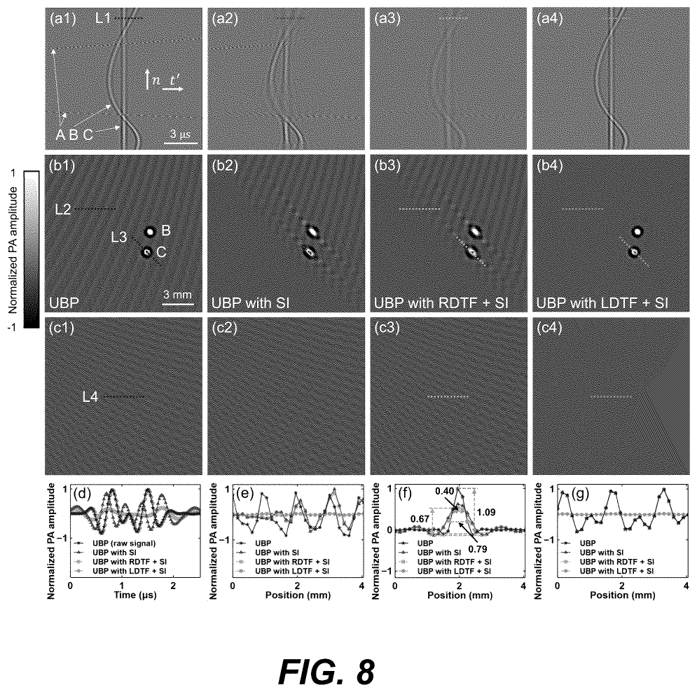

FIGS. 8, 9, 10, and 11 illustrate example experimental data in accordance with some embodiments. Turning to FIG. 8, examples of spatiotemporal antialiasing for the image reconstruction of point sources are shown. Panels (a1), (a2), (a3), and (a4) illustrate the final spatiotemporal signals used for reconstructions in a single sub-domain, D₁, using universal backprojection, universal backprojection with spatial interpolation, universal backprojection and radius-dependent temporal filtering and spatial interpolation (indicated in FIGS. 8-11 as “RDTF”), and universal backprojection and location-dependent temporal filtering and spatial interpolation (e.g., using the techniques shown in and described above in connection with FIG. 6). Note that techniques for performing radius-dependent temporal filtering are shown in and described in U.S. patent application Ser. No. 17/090,752, filed Nov. 5, 2020, which is incorporated by reference herein in its entirety. Panels (b1), (b2), (b3), and (b4) illustrate reconstructions of sub-domain D₁ using the signals shown in (a1), (a2), (a3), and (a4). Panels (c1), (c2), (c3), and (c4) illustrate images of a sub-domain D₂ (i.e., a sub-domain other than the sub-domain in which the point source was located) using the signals shown in (a1), (a2), (a3), and (a4). As illustrated in panel (a1), the spatiotemporal signals using universal backprojection alone include dotted lines that correspond to aliasing in the signal domain. This aliasing yields inaccuracies in the reconstructed images in both the sub-domain D₁ and in sub-domain D₂. For examples, lines L₂ and L₃ in panels (b1) and (b2) correspond to aliasing artifacts that originate from the aliasing shown in panel (a1). When universal backprojection is used with spatial interpolation and/or with radius-dependent temporal filtering (e.g., using the signals of panels (a2) and (a3), respectively), the aliased signals are not completely removed. Moreover, the true signals are blurred, resulting in reduced spatial resolution. Referring to panel (a4), using the location-dependent temporal filtering and location-dependent spatial interpolation techniques disclosed herein, the dotted signal corresponding to the artifact is reduced relative to that of panels (a1), (a2), and (a3). Moreover, the reconstructed signals shown in (b4) and (c4) illustrate that the true signals have increased resolution. Note that the mitigation of artifacts in reconstruction of a sub-domain other than the sub-domain in which the point source is located is also increased, as shown in panel (c4).

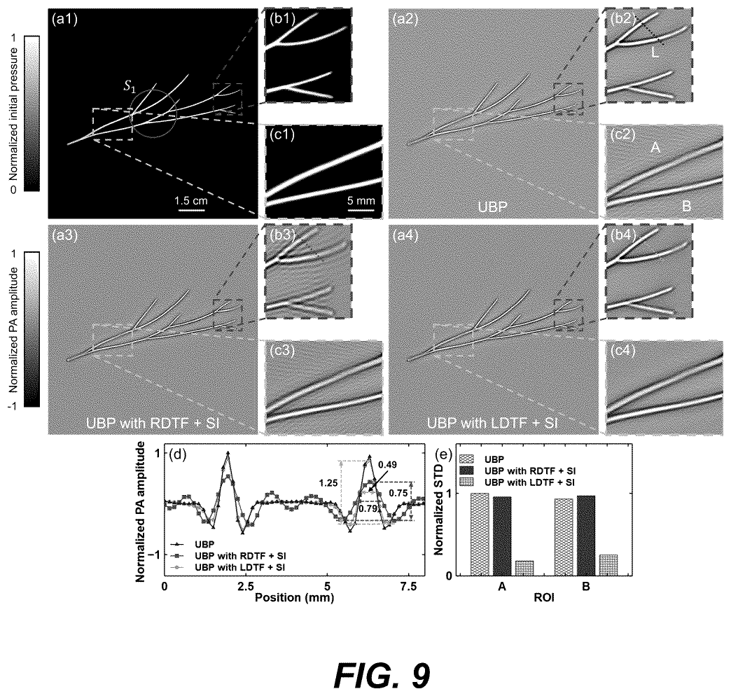

Turning to FIG. 9, an example of application of spatiotemporal antialiasing to the image reconstruction of a numerical phantom with simple blood vessel structures is illustrated in accordance with some embodiments. Panel (a1) illustrates a numerical phantom consisting of simple blood vessel structures. The one-way Nyquist zone S₁ with a radius of 13.6 mm is illustrated. Panels (b1) and (c1) illustrate closeup subsets of the simple numerical phantom, with dashed lines illustrating the portions of (a1) that correspond to panels (b1) and (c1). Panels (a2)-(c2), (a3)-(c3), and (a4)-(c4) illustrate images of the simple numerical phantom reconstructed using universal backprojection alone, universal backprojection with radius-dependent temporal filtering and spatial interpolation (denoted as RDTF in panels (a3)-(c3)), and with universal backprojection with location-dependent temporal filtering and spatial interpolation, respectively. Panel (d) illustrates comparisons of the values along a line L that is selected for comparisons of the three spatiotemporal filtering methods (line L is indicated in panel (b2)). As illustrated in panel (d), the full-width at half maximum (FWHM) of one main lobe is 0.79 mm for universal backprojection and the RDTF technique, and is 0.49 mm for universal backprojection with the location-dependent spatiotemporal filtering technique described herein. Note that the lower main lobe FWHM associated with the location-dependent spatiotemporal filtering technique corresponds to increased spatial resolution when utilizing location-dependent spatiotemporal filtering relative to either backprojection or radius-dependent spatiotemporal filtering. Panel (e) illustrates comparisons of the standard deviations (STDs) of pixel values in regions A and B marked in panel (c2). Note that the STDs associated with regions A and B are smaller with location-dependent temporal filtering and spatial interpolation, indicating the reduction in artifacts with the techniques disclosed herein.

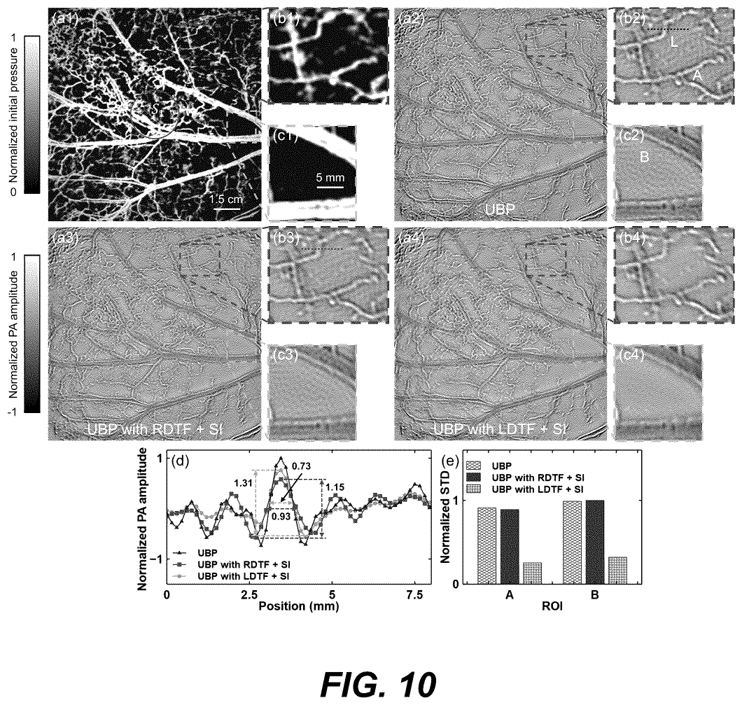

Turning to FIG. 10, an example of application of spatiotemporal antialiasing to the image reconstruction of a numerical phantom with complex blood vessel structures is illustrated in accordance with some embodiments. Panels (a1)-(c1) illustrate the numerical phantom of complex blood vessels (in panel (a1)) and close up subsets of the image of (a1) in panels (b1) and (c1). Panels (a2)-(c2), (a3)-(c3), and (a4)-(c4) illustrate images of the simple numerical phantom reconstructed using universal backprojection alone, universal backprojection with radius-dependent temporal filtering and spatial interpolation (denoted as RDTF in panels (a3)-(c3)), and with universal backprojection with location-dependent temporal filtering and spatial interpolation, respectively. Panel (d) illustrates comparisons of the values along the line L marked in panel (b2) for the three spatiotemporal filtering techniques. The FWHM of the dominant lobe is 0.93 mm for universal backprojection and the RDTF technique, and is 0.73 mm for universal backprojection with the location dependent temporal filtering and spatial interpolation technique described herein. The amplitudes are 1.15 and 1.31, respectively. Note that the lower FWHM and higher amplitude for the universal backprojection with the location dependent temporal filtering and spatial interpolation technique described herein indicates higher spatial resolution and less blurring of the signal of interest. Panel (e) illustrates comparisons of the standard deviations (STDs) of pixel values in regions A and B marked in panels (b2) and (c2), respectively. Note that the STDs associated with regions A and B are smaller with location-dependent temporal filtering and spatial interpolation, indicating the reduction in artifacts with the techniques disclosed herein.

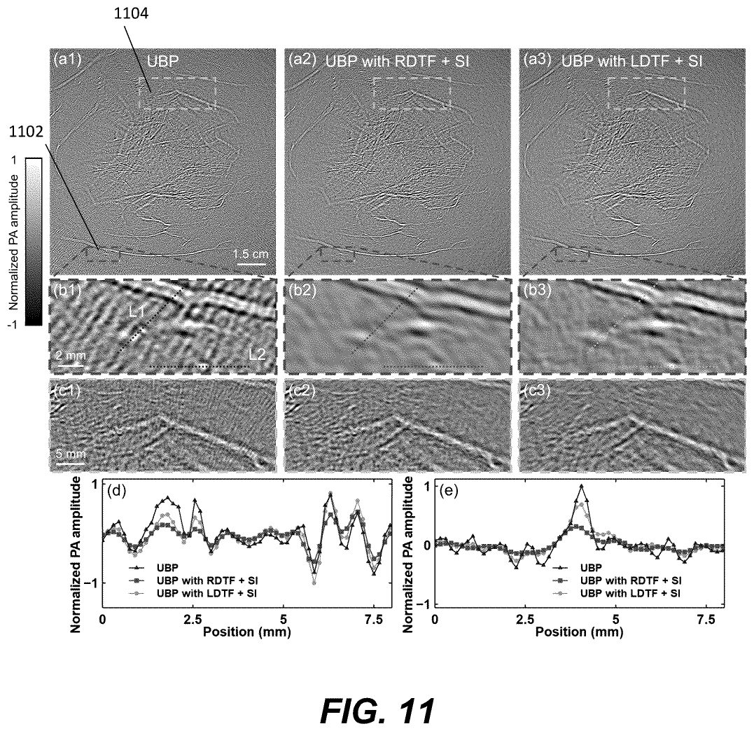

Turning to FIG. 11, an example of application of spatiotemporal antialiasing to human breast imaging in vivo is illustrated in accordance with some embodiments. Panels (a1)-(a3) illustrate images of a human breast cross section in vivo reconstructed using universal backprojection, universal backprojection with radius-dependent spatiotemporal filtering, and universal backprojection using location-dependent temporal filtering and spatial interpolation as described herein, respectively. Panels (b1), (b2), and (b3) correspond to closeups of reconstructions for a first sub-domain 1102, and panels (c1), (c2), and (c3) correspond to reconstructions for a second sub-domain 1104. Panels (d) and (e) depict amplitudes along lines L₁ and L₂ (marked in panel (b1) to compare the three spatiotemporal filtering techniques. As illustrated in panels (d) and (e), location-dependent temporal filtering and spatial interpolation results in relatively high amplitudes for features of interest and smoother values for other regions, indicative of high spatial resolution and reduced artifacts using the spatiotemporal filtering techniques described herein.

Modifications, additions, or omissions may be made to any of the above-described embodiments without departing from the scope of the disclosure. Any of the embodiments described above may include more, fewer, or other features without departing from the scope of the disclosure. Additionally, the steps of described features may be performed in any suitable order without departing from the scope of the disclosure. Also, one or more features from any embodiment may be combined with one or more features of any other embodiment without departing from the scope of the disclosure. The components of any embodiment may be integrated or separated according to particular needs without departing from the scope of the disclosure.

It should be understood that certain aspects described above can be implemented in the form of logic using computer software in a modular or integrated manner. Based on the disclosure and teachings provided herein, a person of ordinary skill in the art will know and appreciate other ways and/or methods to implement the present invention using hardware and a combination of hardware and software.

Any of the software components or functions described in this application, may be implemented as software code using any suitable computer language and/or computational software such as, for example, Java, C, C#, C++ or Python, LabVIEW, Mathematica, or other suitable language/computational software, including low level code, including code written for field programmable gate arrays, for example in VHDL. The code may include software libraries for functions like data acquisition and control, motion control, image acquisition and display, etc. Some or all of the code may also run on a personal computer, single board computer, embedded controller, microcontroller, digital signal processor, field programmable gate array and/or any combination thereof or any similar computation device and/or logic device(s). The software code may be stored as a series of instructions, or commands on a CRM such as a random access memory (RAM), a read only memory (ROM), a magnetic media such as a hard-drive or a floppy disk, or an optical media such as a CD-ROM, or solid stage storage such as a solid state hard drive or removable flash memory device or any suitable storage device. Any such CRM may reside on or within a single computational apparatus, and may be present on or within different computational apparatuses within a system or network. Although the foregoing disclosed embodiments have been described in some detail to facilitate understanding, the described embodiments are to be considered illustrative and not limiting. It will be apparent to one of ordinary skill in the art that certain changes and modifications can be practiced within the scope of the appended claims.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

What is claimed is:

1. A photoacoustic computed tomography method, comprising:
acquiring photoacoustic data recorded by one or more data acquisition devices of a photoacoustic computed tomography system;
applying location-based temporal filtering to the photoacoustic data acquired, wherein applying the location-based temporal filtering is on a sub-domain by sub-domain basis for a plurality of sub-domains, the plurality of sub-domains when aggregated forming an entire imaging domain; and
reconstructing one or more photoacoustic images from the filtered photoacoustic data.

2. The photoacoustic computed tomography method of claim 1, further comprising applying spatial interpolation after applying the location-based temporal filtering.

3. The photoacoustic computed tomography method of claim 2, wherein the spatial interpolation is performed on the sub-domain by sub-domain basis for the plurality of sub-domains.

4. The photoacoustic computed tomography method of claim 3, wherein the temporal filtering mitigates aliasing prior to the spatial interpolation.

5. The photoacoustic computed tomography method of claim 1, wherein applying the location-based temporal filtering comprises:
determining an upper cutoff frequency for each sub-domain; and
applying one or more lowpass filters associated with the upper cutoff frequency.

6. The photoacoustic computed tomography method of claim 5, wherein the one or more lowpass filters associated with the upper cutoff frequency comprise a plurality of lowpass filters each having a different cutoff frequency less than the upper cutoff frequency, and wherein applying the location-based temporal filtering comprises:
applying the plurality of lowpass filters to the photoacoustic data; and
recentering the filtered photoacoustic data for different image subdomains.

7. The photoacoustic computed tomography method of claim 5, wherein the upper cutoff frequency is selected such that a Nyquist criterion is satisfied for each sub-domain.

8. The photoacoustic computed tomography method of claim 5, wherein the upper cutoff frequency for a given sub-domain is determined based on relative locations of transducer elements, a center of the sub-domain, and points on a boundary of the sub-domain.

9. The photoacoustic computed tomography method of claim 5, wherein a point source is outside of a sub-domain, and wherein the upper cutoff frequency for the sub-domain is further based on relative locations of transducer elements, a center of the sub-domain and the point source.

10. The photoacoustic computed tomography method of claim 1, wherein a general source causes reflections indicated in the photoacoustic data, and wherein the method further comprises:
performing an initial reconstruction without anti-aliasing to generate a reconstructed image;
obtaining a set of point source candidates based on the reconstructed image;
dividing the entire imaging domain into a set of squares;
randomly selecting one point source in each square of the set of squares as a point source;
applying the location-based filtering for each sub-domain based on the selected point sources to generate an image;
repeating the random selection of one point source in each square and applying the location-based filtering for each sub-domain at least one other time to obtain multiple images; and
generating a final reconstructed image based on an average of the multiple images.

11. The photoacoustic computed tomography method of claim 10, wherein the initial reconstruction is performed using universal back projection.

12. The photoacoustic computed tomography method of claim 10, wherein the set of point source candidates is obtained by applying a threshold to the reconstructed image.

13. The photoacoustic computed tomography method of claim 1, wherein universal back projection is used to reconstruct the one or more photoacoustic images.

14. A non-transitory computer readable media, the non-transitory computer readable media, when read by one or more processors, is configured to perform operations comprising:
acquiring photoacoustic data recorded by one or more data acquisition devices of a photoacoustic computed tomography system;
applying location-based temporal filtering to the photoacoustic data acquired, wherein applying the location-based temporal filtering is on a sub-domain by sub-domain basis for a plurality of sub-domains, the plurality of sub-domains when aggregated forming an entire imaging domain; and
reconstructing one or more photoacoustic images from the filtered photoacoustic data.

15. The non-transitory computer readable media of claim 14, wherein the operations further comprise applying spatial interpolation after applying the location-based temporal filtering.

16. The non-transitory computer readable media of claim 15, wherein the spatial interpolation is performed on the sub-domain by sub-domain basis for the plurality of sub-domains.

17. The non-transitory computer readable media of claim 16, wherein the temporal filtering mitigates aliasing prior to the spatial interpolation.

18. The non-transitory computer readable media of claim 14, wherein applying the location-based temporal filtering comprises:
determining an upper cutoff frequency for each sub-domain; and
applying one or more lowpass filters associated with the upper cutoff frequency.

19. The non-transitory computer readable media of claim 18, wherein the one or more lowpass filters associated with the upper cutoff frequency comprise a plurality of lowpass filters each having a different cutoff frequency less than the upper cutoff frequency, and wherein applying the location-based temporal filtering comprises:
applying the plurality of lowpass filters to the photoacoustic data; and
recentering the filtered photoacoustic data.

20. The non-transitory computer readable media of claim 18, wherein the upper cutoff frequency is selected such that a Nyquist criterion is satisfied for each sub-domain.

21. The non-transitory computer readable media of claim 18, wherein the upper cutoff frequency for a given sub-domain is determined based on relative locations of transducer elements, a center of the sub-domain, and points on a boundary of the sub-domain.

22. The non-transitory computer readable media of claim 18, wherein a point source is outside of a sub-domain, and wherein the upper cutoff frequency for the sub-domain is further based on relative locations of transducer elements, a center of the sub-domain and the point source.

23. The non-transitory computer readable media of claim 14, wherein a general source causes reflections indicated in the photoacoustic data, and wherein the operations further comprise:
performing an initial reconstruction without anti-aliasing to generate a reconstructed image;
obtaining a set of point source candidates based on the reconstructed image;
dividing the entire imaging domain into a set of squares;
randomly selecting one point source in each square of the set of squares as a point source;
applying the location-based filtering for each sub-domain based on the selected point sources to generate an image;
repeating the random selection of one point source in each square and applying the location-based filtering for each sub-domain at least one other time to obtain multiple images; and
generating a final reconstructed image based on an average of the multiple images.
